(19) United States
(12) Patent Application Publication
(10) Pub. No.: US 2024/0130999 A1
(43) Pub. Date: Apr. 25, 2024

(54) Inhibition of SLC4A4 in the Treatment of Cancer

(71) Applicants: VIB VZW, Gent (BE); KATHOLIEKE UNIVERSITEIT LEUVEN, Leuven (BE)

(72) Inventors: Massimiliano Mazzone, Kessel-Lo (BE); Federica Cappellesso, Leuven (BE); Federico Virga, Leuven (BE)

(21) Appl. No.: 18/546,682

(22) PCT Filed: Feb. 17, 2022
(86) PCT No.: PCT/EP2022/053957

§ 371 Date: Aug. 16, 2023

(30) Foreign Application Priority Data
Feb. 17, 2021 (EP) 21157705.1

(51) Int. Cl. A61K 31/26; A61K 38/46; A61K 39/395; A61P 35/00; C12N 15/113

(52) U.S. Cl. CPC A61K 31/26; A61K 38/465; A61K 39/3955; A61P 35/00; C12N 15/1138

(57) ABSTRACT

The invention relates to the application of inhibition of SLC4A4 (Solute Carrier Family 4 member 4) in the treatment of cancer. This either as monotherapy (such as for treating cancer refractive or poorly responding to immunotherapy) or as combination therapy in conjunction with an immunotherapeutic compound (such as for treating cancers poorly responding or refractive to immunotherapy). In particular, inhibition of SLC4A4 is capable of restoring response to immunotherapy such as immune checkpoint inhibitor therapy.

### CROSS-REFERENCE TO RELATED APPLICATIONS

This application is a national phase entry under 35 U.S.C. § 371 of International Patent Application PCT/EP2022/053957, filed Feb. 17, 2022, designating the United States of America and published in English as International Patent Publication WO 2022/175392 on Aug. 25, 2022, which claims the benefit under Article 8 of the Patent Cooperation Treaty to European Patent Application Serial No. 21157705.1, filed Feb. 17, 2021, the entireties of which are hereby incorporated by reference.

### STATEMENT ON EUROPEAN FUNDING

The project leading to this application has received funding from the European Union's Horizon 2020 research and innovation program under grant agreement No 766214.

### FIELD OF THE INVENTION

The invention relates to the application of inhibition of SLC4A4 (Solute Carrier Family 4 member 4) in the treatment of cancer. This either as monotherapy (such as for treating cancer refractive or poorly responding to immunotherapy) or as combination therapy in conjunction with an immunotherapeutic compound (such as for treating cancers poorly responding or refractive to immunotherapy). In particular, inhibition of SLC4A4 is capable of restoring response to immunotherapy such as immune checkpoint inhibitor therapy.

### BACKGROUND OF THE INVENTION

Solute carrier proteins (SLCs) form a very diverse group of membrane transporters with over 400 members classified in 65 families.

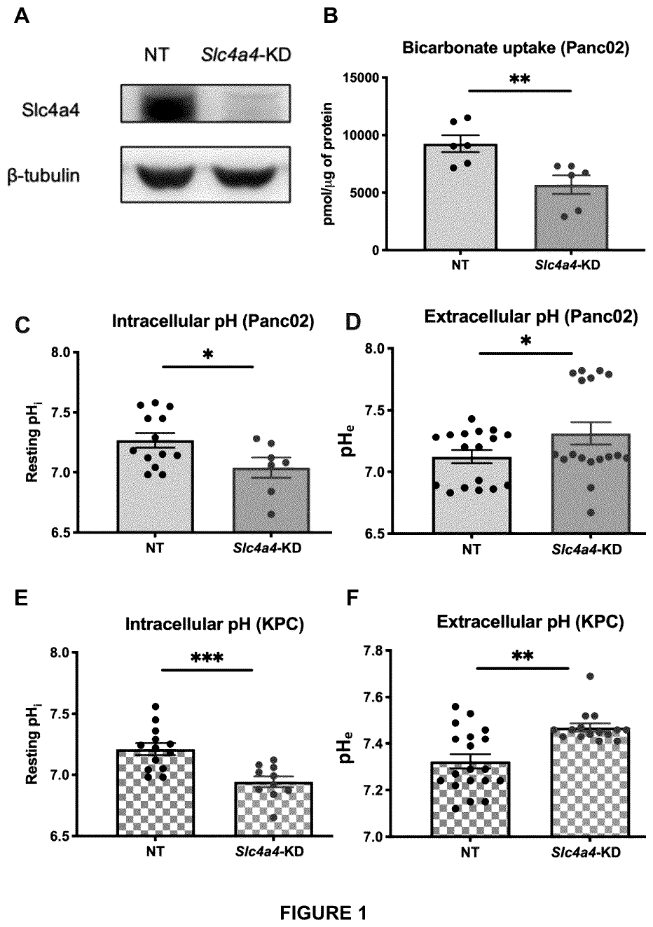

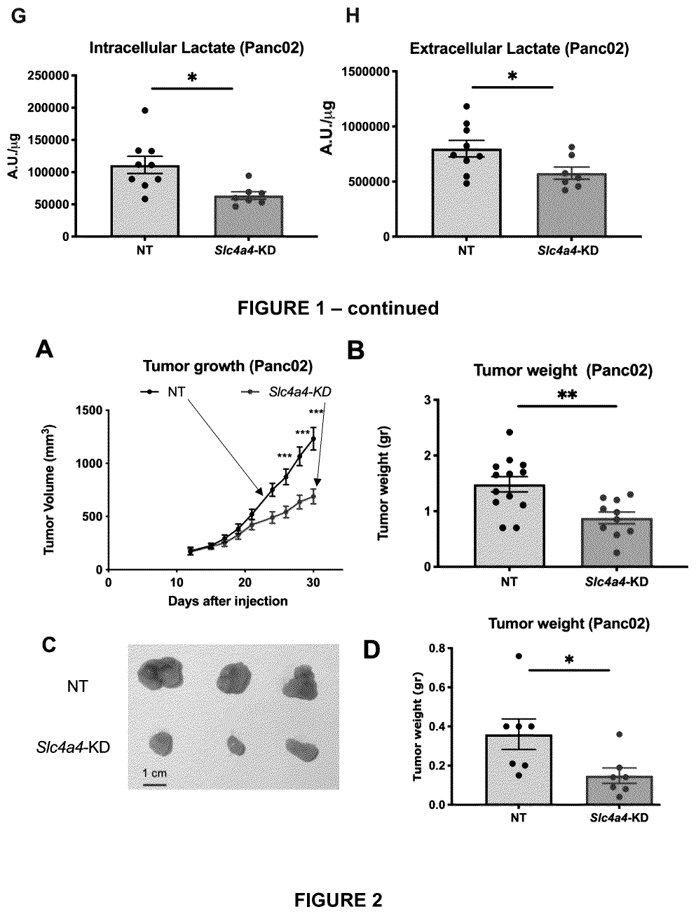

One group of SLCs are the bicarbonate transporters which can be subdivided further according to whether an individual bicarbonate transporter is sodium independent or sodium driven, and according to whether an individual bicarbonate transporter is an acid loader, an acid extruder, or whether its acidification effect is variable or unclear. SLC4A4 is one of the several sodium driven, acid extruding bicarbonate transporters (others including SLC4A6, SLC4A7, SLC4A8, SLC4A9 and SLC4A10) (FIG. 1A of McIntyre et al. 2015, Cancer Res 76:3744-3755). SLCs are reviewed in e.g. Parker & Boron 2013 (Physiol Rev 93:803-959).

SLC4A4 (Solute Carrier Family 4 member 4, also known as NBC1; US6096517) is protecting cells against intracellular acidosis (low intracellular pH, pHi). S0859 was developed as inhibitor of Na-driven bicarbonate transporters, but is not specific (Heidtmann et al. 2015, Eur J Pharmacol 762:344-349). DIDS (4,4′-diisothiocyano-2,2′-stilbenedisulfonic acid) is another non-specific inhibitor. Polyclonal Ab preps (including inhibitory and excitatory IgG preps) have been described (De Giusti et al. 2011, Br J Pharmacol 164:1976-1989; Khandoudi et al. 2001, Cardiovasc Res 52:387-396). The cryoEM structure of hSLC4A4 has been determined (Huynh et al. 2018, Nat Commun 9:900).

shRNA knockdown (kd) of SLC4A4 in cancer cell lines (MDA-MB-231 breast cancer, expressing high levels of SLC4A4) resulted in strong impact on cell proliferation, migration and invasion (Parks & Pouyssegur 2015, J Cell Physiol 230:1954-1963). SLC4A4 was suggested as one of four prognostic markers/therapeutic targets of colorectal cancer (Bian et al. 2019, Oncol Lett 18:5043-5054). SLC4A4 kd and SLC4A9 kd interfere with spheroid growth of LS174 (colorectal cancer cells) and knockdown of SLC4A9 dramatically reduces tumor xenograft formation (McIntyre et al 2016—Cancer Res 76:3744-3755). Genetic disruption of NBCn1 (SLC4A7) delayed breast cancer development: tumor latency was ˜50% increased while tumor growth rate was ˜65% reduced in NBCn1 KO compared with wild-type (WT) mice. Breast cancer histopathology in NBCn1 KO mice differed from that in WT mice and included less aggressive tumor types (Lee et al. 2016—Oncogene 35:2112-2122).

Tumor acidity is emerging as one of the many regulators of anti-tumor immunity. A low pH within the tumor microenvironment (TME) can affect the functioning of immune cells (Pilon-Thomas et al. 2016, Cancer Res 76:1381-1390) and possibly affect the therapeutic efficacy of immune checkpoint inhibitors (Pilon-Thomas et al. 2016, Cancer Res 76:1381-1390; Renner et al. 2019, Cell Rep 29:135-150). Tackling pH dysregulation may improve anti-tumor immune responses (Pilon-Thomas et al. 2016, Cancer Res 76:1381-1390; Renner et al. 2019, Cell Rep 29:135-150; Brand et al. 2016, Cell Metab 24:657-671). The role of bicarbonate transporters in this process has not been assessed, and the existence of multiple such transporters probably redundant in function is considered a complicating factor.

Currently nothing is known on the effect of inhibition of SLC4A4 in an in vivo cancer setting, such as in in vivo models of pancreatic adenocarcinoma (PDAC) for which no satisfactory pharmacological treatment (including immune checkpoint inhibitors) is currently available. Further in particular, currently nothing is known on the effect of inhibition of SLC4A4 towards in vivo immune response, such as immune responses modified by checkpoint inhibitors. Indeed research on cancer cell lines cannot provide such information as the immune compartment is absent in such assays.

On the other hand, and although the introduction of immune checkpoint inhibitors has revolutionized clinical practice of cancer treatment, it is clear that only a subset of cancer patients (including within the same cancer type) is responding to immune checkpoint inhibitor therapy. There is thus a continued need to provide ways to increase the success rate of therapy in cancer refractive to immune checkpoint inhibitor therapy, and, overall, to increase the success rate of immune checkpoint inhibitor therapy.

### SUMMARY OF THE INVENTION

The invention in one aspect relates to an inhibitor of Solute Carrier Family 4 member 4 (SLC4A4) for use in treating or inhibiting cancer, or for use in inhibiting progression, relapse or metastasis of cancer, wherein the cancer is poorly responding to or resistant to immunotherapy or to therapy comprising an immunotherapeutic compound or agent.

In a further aspect, the invention relates to an inhibitor of Solute Carrier Family 4 member 4 (SLC4A4) for use in treating or inhibiting pancreatic cancer, or for use in inhibiting progression, relapse or metastasis of pancreatic cancer.

In one embodiment, the inhibitor of SLC4A4 is for use according to these aspects in combination with immunotherapy.

In a further embodiment to these aspects, the inhibitor of SLC4A4 is a specific inhibitor of SLC4A4. In particular, the specific inhibitor of SLC4A4 is a DNA nuclease specifically knocking out or disrupting SLC4A4, an RNase specifically targeting SLC4A4, or an inhibitory oligonucleotide specifically targeting SLC4A4. In particular, the specific inhibitor of SLC4A4 is a pharmacological inhibitor specifically inhibiting SLC4A4 and is selected from the group consisting of a polypeptide comprising an immunoglobulin variable domain, a monoclonal antibody or a fragment thereof, an alpha-body, a nanobody, an intrabody, an aptamer, a DARPin, an affibody, an affitin, an anticalin, a monobody, a bicyclic peptide, a PROTAC, or a LYTAC.

When referring above to immunotherapy, this may in particular be immunotherapy comprising therapy with one or two immune checkpoint inhibitors. In particular, such two immune checkpoint inhibitors are each inhibiting a different immune checkpoint or a different immune checkpoint-ligand interaction.

In a further aspect, the invention relates to an immunotherapeutic compound or agent for use in treating or inhibiting cancer, or for use in inhibiting progression, relapse or metastasis of cancer, in combination with an inhibitor of SLC4A4. In one embodiment to this aspect, the inhibitor of SLC4A4 is a specific inhibitor of SLC4A4.

In a further aspect, the invention relates to a combination of an inhibitor of Solute Carrier Family 4 member 4 (SLC4A4) and an immunotherapeutic compound or agent; as well as to composition comprising such combination. In one embodiment, such combination or composition comprises at least one immune checkpoint inhibitor. In a further embodiment, such combination or composition is for use as a medicine, such as for use in treating or inhibiting cancer, or for use in inhibiting progression, relapse or metastasis of cancer.

### BRIEF DESCRIPTION OF THE DRAWINGS

### FIGS. 1A-1H

FIG. 1A. Slc4a4 expression levels assessed by Western Blot analysis in NT and Slc4a4-KD Panc02 cells

FIG. 1B. ¹⁴C-Bicarbonate uptake in NT and Slc4a4-KD Panc02 cells (n=6).

FIG. 1C. Intracellular pH levels of NT (n=13) and Slc4a4-KD (n=7) Panc02 cells.

FIG. 1D. Extracellular pH levels of NT (n=17) and Slc4a4-KD (n=17) Panc02 cells.

FIG. 1E. Intracellular pH levels of NT (n=13) and Slc4a4-KD (n=10) KPC cells.

FIG. 1F. Extracellular pH levels of NT (n=19) and Slc4a4-KD (n=15) KPC cells.

FIG. 1G, FIG. 1H. Intracellular (FIG. 1G) and extracellular (FIG. 1H) lactate levels measured by LC/MS analysis in NT and Slc4a4-KD Panc02 cells (n=3). Data are normalized by protein content.

P-value was assessed by unpaired, two-tailed Student's t-test (FIGS. 1B,1C, 1E, 1G, 1H) and paired two-tailed Student's t-test (FIGS. 1D,1F). Statistical analysis: * P<0.05; ** P<0.005; *** P<0.0005; graphs show mean±SEM.

See Example 2 for more details.

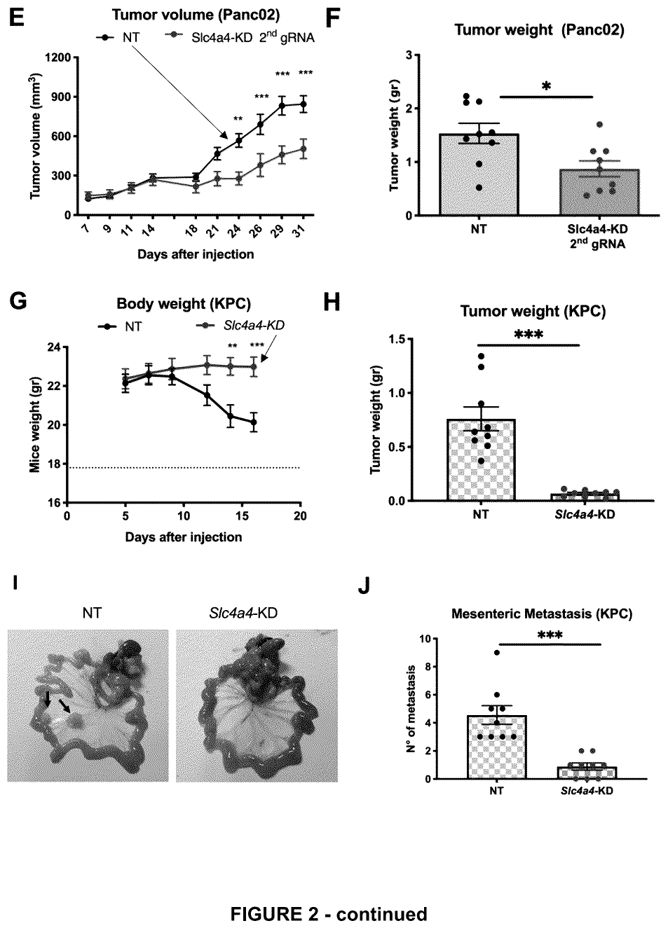

### FIGS. 2A-2J

FIGS. 2A-2C. Tumor growth (FIG. 2A), tumor weight (FIG. 2B) and representative picture (FIG. 2C) of NT (n=13) and Slc4a4-KD (n=10) Panc02 subcutaneous tumors.

FIG. 2D. Tumor weight of NT (n=7) and Slc4a4-KD (n=7) Panc02 orthotopic tumors.

FIGS. 2E, 2F. Tumor growth (FIG. 2E) and tumor weight (FIG. 2F) of NT (n=9) and Slc4a4-KD 2^{nd} gRNA (n=9) Panc02 subcutaneous tumors.

FIGS. 2G-2J. Body weight (FIG. 2G), tumor weight (FIG. 2H), mesenteric metastasis quantification (FIG. 2J) and representative pictures (FIG. 2I) of mice injected with NT (n=9) and Slc4a4-KD (n=9) KPC orthotopic tumors.

P-value was assessed by unpaired, two-tailed Student's t-test (FIGS. 2B, 2D, 2F, 2H, 2J) and two-way ANOVA with Sidak's multiple comparison test (FIGS. 2A, 2E, 2G). Statistical analysis: * P<0.05; ** P<0.005; *** P<0.0005; graphs show mean±SEM.

See Examples 2 and 3 for more details.

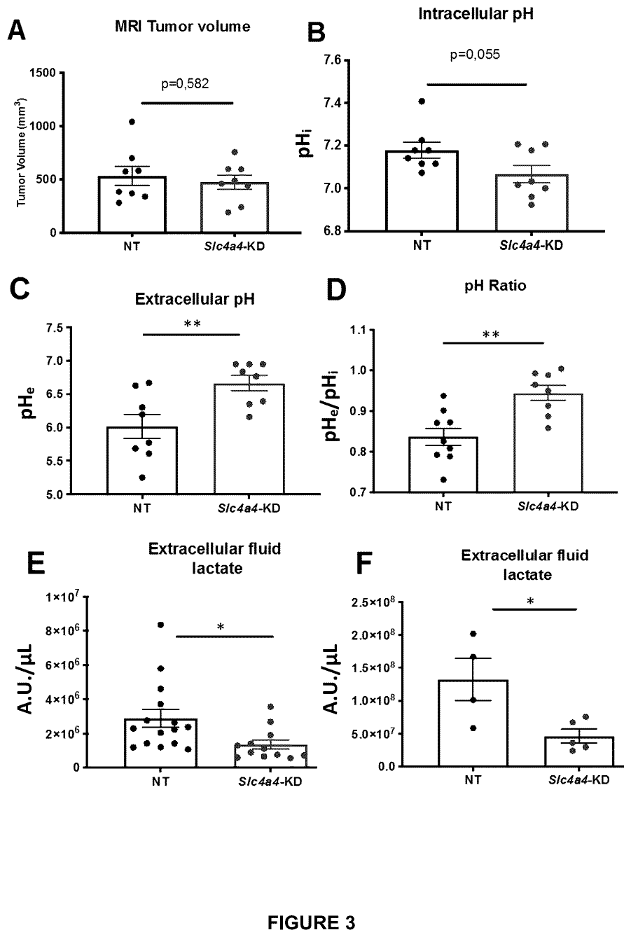

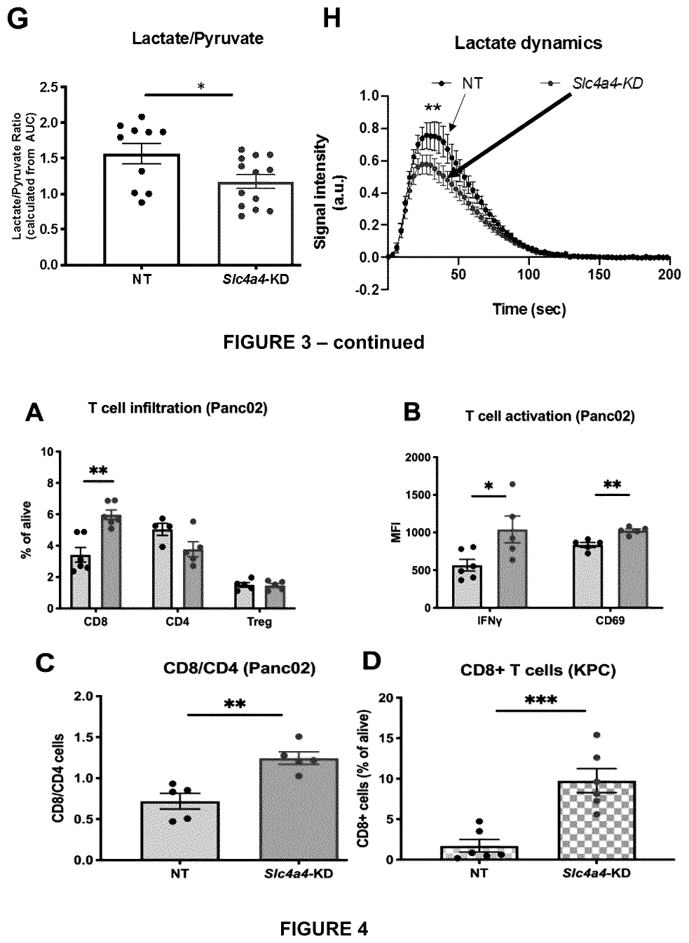

### FIGS. 3A-3H

FIG. 3A. Tumor volume assessed by magnetic resonance imaging (MRI) of NT (n=8) and Slc4a4-KD (n=8) Panc02 subcutaneous tumors

FIGS. 3B-3D. Intracellular (FIG. 3B), extracellular pH (FIG. 3C) and pH ratio (FIG. 3D) of NT (n=8) and Slc4a4-KD (n=8) Panc02 subcutaneous tumors assessed by ³¹P-MRS.

FIGS. 3E, 3F. Lactate concentration measured by LC/MS in extracellular fluid of NT (n=15) and Slc4a4-KD (n=12) Panc02 subcutaneous tumors (FIG. 3E) and NT (n=4) and Slc4a4-KD (n=5) KPC orthotopic tumors (FIG. 3F).

FIG. 3G, 3H. Lactate to pyruvate ratio (FIG. 3G) and lactate dynamics (FIG. 3H) of NT (n=8) and Slc4a4-KD (n=8) Panc02 subcutaneous tumors assessed by MRI with hyperpolarized lactate.

P-value was assessed by unpaired, two-tailed Student's t-test (FIG. 3A-3G). Area under the curve (FIG. 3H) Statistical analysis: * P<0.05; ** P<0.005; *** P<0.0005; graphs show mean±SEM.

See Example 3 for more details.

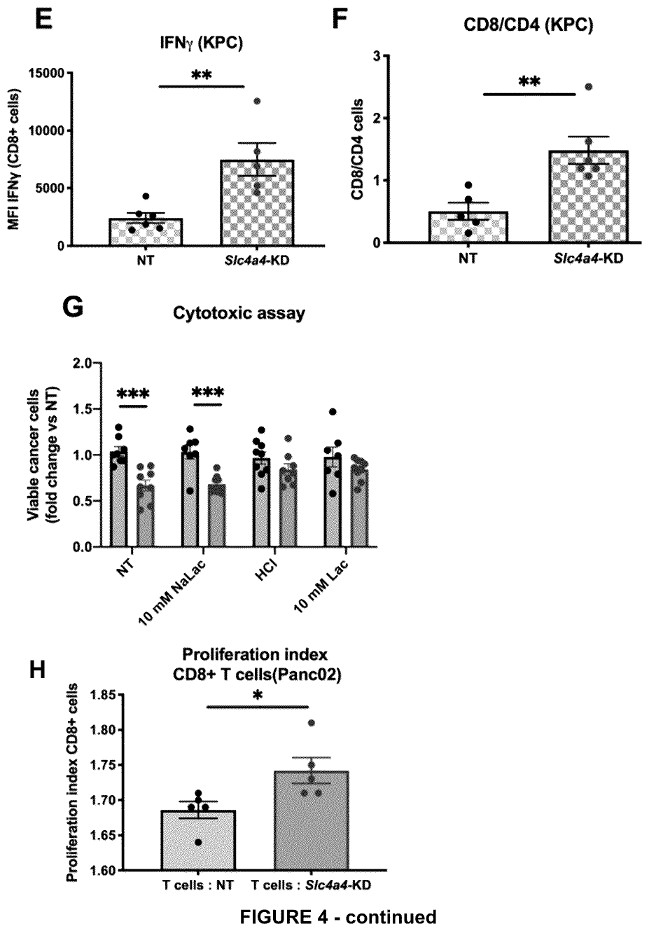

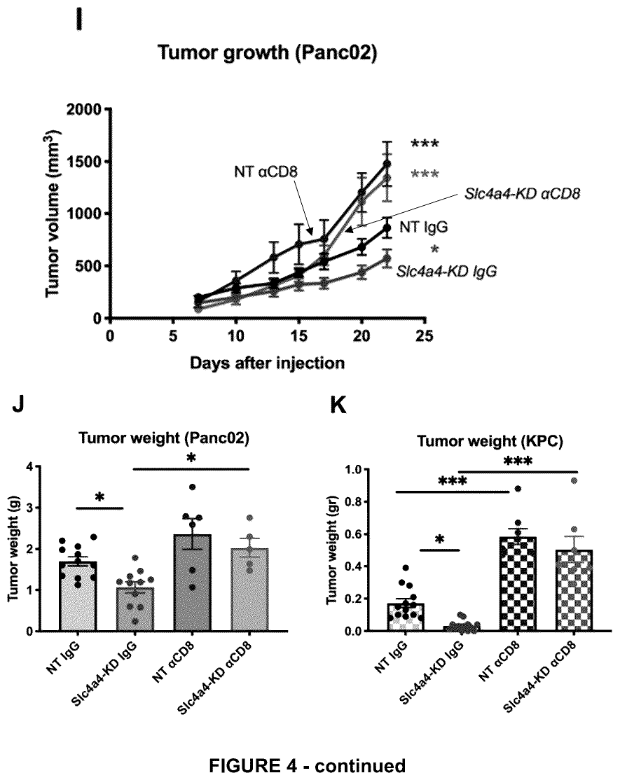

### FIGS. 4A-4K

FIG. 4A. T cell infiltration analyzed by FACS in Panc02 subcutaneous tumors. CD8⁺ cells NT (n=6) and Slc4a4-KD (n=6), CD4⁺ cells NT (n=4) and Slc4a4-KD (n=5), Foxp3⁺ cells NT (n=5) and Slc4a4-KD (n=5).

FIG. 4B. T cell activation analyzed by FACS in Panc02 subcutaneous tumors. MFI of IFNγ in CD8⁺ cells NT (n=6) and Slc4a4-KD (n=5), MFI of CD69 in CD8⁺ cells NT (n=5) and Slc4a4-KD (n=5).

FIG. 4C. CD8/CD4 ratio analyzed by FACS in Panc02 subcutaneous tumors, NT (n=5) and Slc4a4-KD (n=5).

FIG. 4D. FACS analysis of CD8⁺ T cells in KPC orthotopic tumors, NT (n=6) and Slc4a4-KD (n=6).

FIG. 4E. MFI of IFNγ in CD8⁺ cells analyzed by FACS in KPC orthotopic tumors, NT (n=6) and Slc4a4-KD (n=5).

FIG. 4F. CD8/CD4 ratio analyzed by FACS in KPC orthotopic tumors, NT (n=6) and Slc4a4-KD (n=5).

FIG. 4G. Number of viable Panc02-OVA cells co-culture with activated OT-1 T cells in a 1:5 ratio in non-treated T cell medium (NT) or medium supplemented with 10 mM sodium lactate (NaLac), acidified to pH=6.3 (HCl) or supplemented with 10 mM Lactic acid at pH=6.3 (Lac). NT (n=7) and Slc4a4-KD (n=8).

FIG. 4H. Proliferation assay of CD8 T cell cultured with NT (n=5) or Slc4a4-KD (n=5) Panc02 cell conditioned medium.

FIGS. 4I, 4J. Tumor growth (FIG. 4I) and tumor weight (FIG. 4J) of NT and Slc4a4-KD Panc02 tumor subcutaneously injected in CD8 depleted mice (NT IgG n=12, Slc4a4-KD IgG n=11, NT αCD8 n=6, Slc4a4-KD αCD8 n=5).

FIG. 4K. Tumor weight of NT and Slc4a4-KD KPC tumor orthotopically injected in CD8 depleted mice (NT IgG n=12, Slc4a4-KD IgG n=12, NT αCD8 n=8, Slc4a4-KD αCD8 n=7).

FIGS. 4A, 4B, and 4G: per X-axis value, the left bar corresponds to NT Panc02 subcutaneous tumors and the right bar corresponds to Slc4a4-KD Panc02 subcutaneous tumors.

P-value was assessed by unpaired, two-tailed Student's t-test (FIGS. 4A-4F, 4H) and two-way ANOVA with Sidak's multiple comparison test (FIGS. 4G, 4I, 4J, 4K). Statistical analysis: * P<0.05; ** P<0.005; *** P<0.0005; graphs show mean±SEM.

See Example 4 for more details.

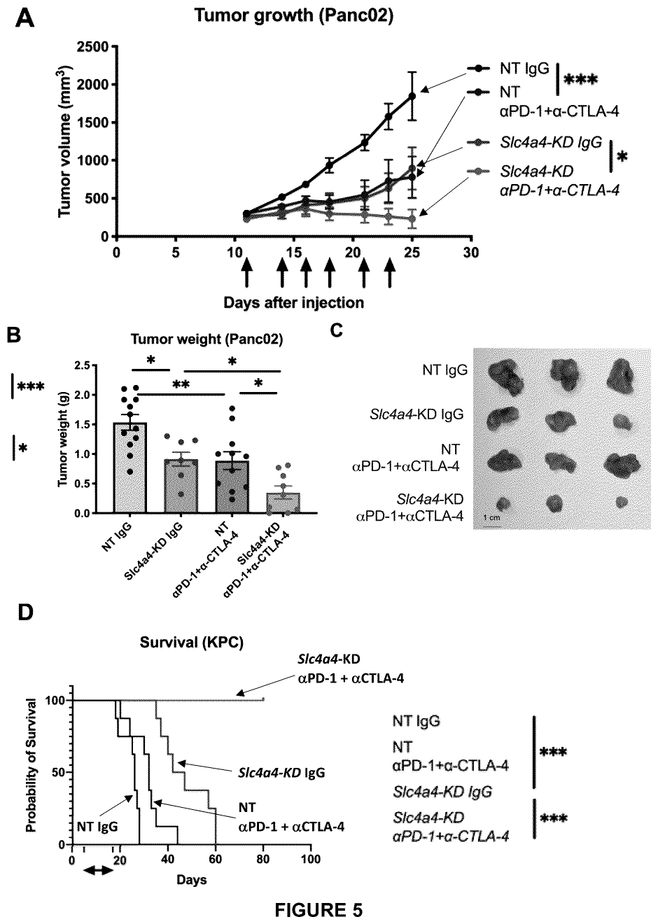

### FIGS. 5A-5D

FIGS. 5A-5C. Tumor growth (FIG. 5A), tumor weight (FIG. 5B) and representative picture (FIG. 5C) of NT and Slc4a4-KD Panc02 subcutaneous tumors treated with anti-PD-1 and anti-CTLA-4. n=8-9 (treatment regimen indicated by the arrows).

FIG. 5D. Survival curve of NT and Slc4a4-KD KPC orthotopic tumors treated with anti-PD-1 and anti-CTLA-4. n=8. Treatment regimen period indicated by the arrow.

P-value was assessed by two-way ANOVA with Tukey's multiple comparison test (FIGS. 5A, 5B) and Long-rank (Mantel-cox) test (FIG. 5D). Statistical analysis: * P<0.05; ** P<0.005; *** P<0.0005; graphs show mean±SEM.

See Example 5 for more details.

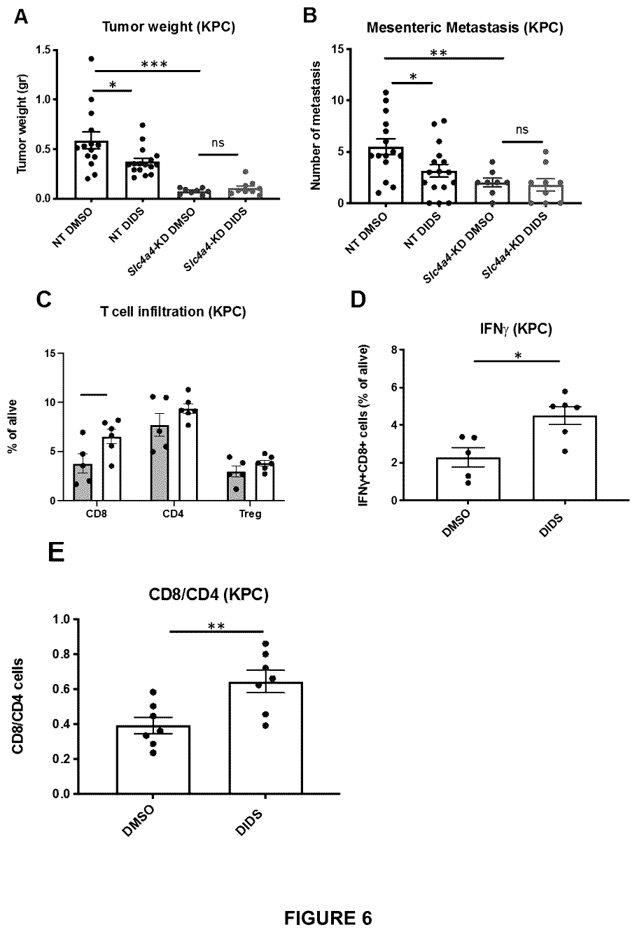

### FIGS. 6A-6E

FIGS. 6A,6B. Tumor weight (FIGS. 6A) and tumor growth (FIGS. 6B) of NT and Slc4a4-KD KPC orthotopic tumors treated with 15 mg/kg DIDS bi-daily from day 5 to day 15 (NT DMSO n=14, NT DIDS n=16, Slc4a4-KD DMSO n=8, Slc4a4-KD DIDS n=9).

FIG. 6C. T cell infiltration analyzed by FACS in KPC orthotopic tumors. CD8⁺, CD4⁺, Treg⁺ cells in DMSO-(n=5) or DIDS-(n=6) treated mice. For each X-axis value: left bar corresponds to DMSO-treated mice and right bar corresponds to DIDS-treated mice.

FIG. 6D. MFI of IFNγ in CD8⁺ cells analyzed by FACS in KPC orthotopic tumors, DMSO-(n=5) or DIDS-(n=6) treated mice.

FIG. 6E. CD8/CD4 ratio analyzed by FACS in KPC orthotopic tumors, DMSO-(n=5) or DIDS-(n=6) treated mice.

P-value was assessed by unpaired, two-tailed Student's t-test (FIGS. 6C, 6D, 6E) and two-way ANOVA with Sidak's multiple comparison test (FIGS. 6A, 6B). Statistical analysis: * P<0.05; ** P<0.005; *** P<0.0005; graphs show mean±SEM.

See Example 6 for more details.

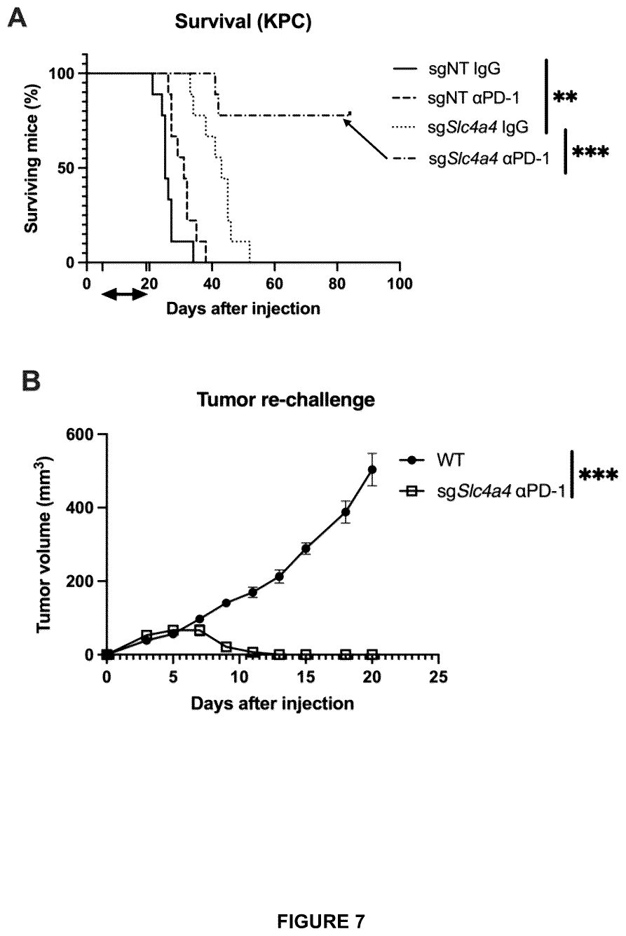

### FIGS. 7A-7B

FIG. 7A. Survival curve of sgNT and sgSlc4a4 orthotopic KPC tumor-bearing mice treated with anti-PD-1. (sgNT IgG n=9, sgNT αPD1 n=9, sgSlc4a4 IgG n=9, sgSlc4a4 αPD1 n=9). Mice were treated 3 times per week up to 6 injections.

FIG. 7B. Growth curves of sgNT subcutaneous KPC tumors injected in mice after the total regression of sgSlc4a4 tumors treated with anti-PD-1 (see sgSlc4a4 αPD1 dotted line panel A) or in WT mice (WT =7, sgSlc4a4 αPD1=7).

P value was assessed by two-way ANOVA with Long-rank (Mantel-cox) test (FIG. 7A) and two-way ANOVA with Tukey's multiple comparison test (FIG. 7B). Statistical analysis: * P<0.05; ** P<0.01; *** P<0.001; graphs show mean±SEM.

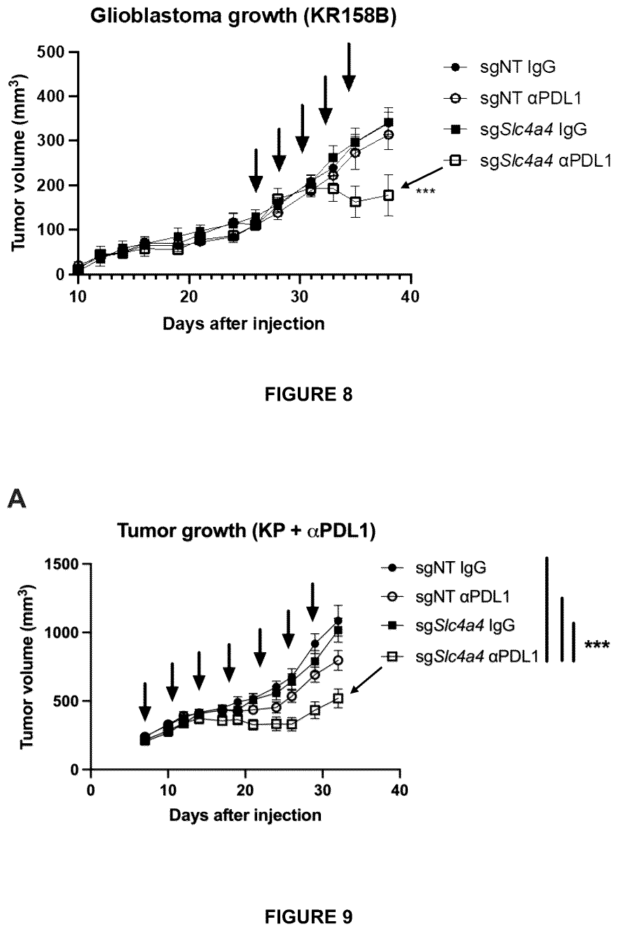

### FIG. 8

Growth of sgNT and sgSlc4a4 subcutaneous glioblastoma KR158B tumors treated with anti-PDL1 (α-PDL1). (sgNT IgG n=7, sgNT αPDL1 n=6, sgSlc4a4 IgG n=6, sgSlc4a4 αPDL1 n=5). Treatment regimen indicated by the arrows.

Statistical analysis: * P<0.05; ** P<0.01; *** P<0.001; graphs show mean±SEM.

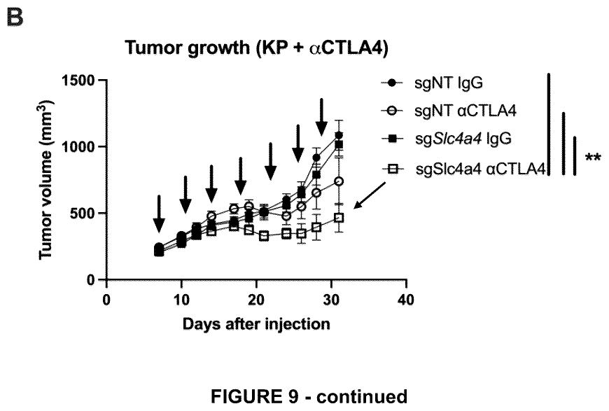

### FIGS. 9A and 9B

FIG. 9A. Growth of sgNT and sgSlc4a4 subcutaneous KP tumors treated with anti-PDL1 (αPDL1). (sgNT IgG n=13, sgNT αPDL1 n=14, sgSlc4a4 IgG n=15, sgSlc4a4 αPDL1 n=14). Treatment regimen indicated by the arrows.

FIG. 9B. Growth of sgNT and sgSlc4a4 subcutaneous KP tumors treated with anti-CTLA4 (αCTLA4). (sgNT IgG n=13, sgNT αCTLA4 n=12, sgSlc4a4 IgG n=15, sgSlc4a4 αCTLA4 n=12). Treatment regimen indicated by the arrows.

P value was assessed with two-way ANOVA with Tukey's multiple comparison test (FIGS. 9A-9B). Statistical analysis: * P≤0.05; ** P<0.01; *** P<0.001; graphs show mean±SEM.

### DETAILED DESCRIPTION

As indicated in the introduction, work on SLC4A4 inhibition in the cancer field so far was limited to effects on growth on cancer cell cultures, not allowing to expect any success as to whether SLC4A4 inhibition would be able to modulate a subject's immune response to any cancer. In addition, such expectation of success of the effect of single SLC4A4 inhibition is further hampered by the existence of other solute carrier proteins having activities redundant with SLC4A4 activity. On the other hand, whereas immune checkpoint inhibitor therapy has revolutionized the field of cancer treatment, clinical experience learned that not all patients, even those having the same cancer, respond to such immune checkpoint inhibitor therapy. It is furthermore not clear if the underlying mechanisms of non-response of different cancer types to immune checkpoint inhibitor therapy are identical for each and any non-responding cancer type. Irrespective thereof, there is a dire need for solutions to increase the response rate to immune checkpoint inhibitor therapy.

In work leading to the current invention as explained in the Figures and Examples, it was first demonstrated that inhibition of SLC4A4 (by genetic knockdown or pharmacologically) is more effective in treating pancreatic cancer in an in vivo model than treatment with a combination of two immune checkpoint inhibitors (anti-PD-1 and anti-CTLA-4). Upon combining inhibition of SLC4A4 with immune checkpoint inhibitors, a synergistic further reduction in pancreatic cancer growth was observed, which translated in an unprecedented increase of overall survival. Inhibition of SLC4A4 as option for treating pancreatic cancer has not been previously explored. Furthermore, inhibition of SLC4A4 was thus demonstrated to i) be effective on its own, and to ii) enhance the efficacy of immune checkpoint inhibitors in treating a cancer known to be largely refractive to such immune checkpoint inhibitor therapy. In addition, it was subsequently demonstrated that inhibition of SLC4A4 in combination with a single immune checkpoint inhibitor (anti-PD1; thus reducing adverse events caused by combining two immune checkpoint inhibitors) is (i) synergistically repressing pancreatic cancer growth and, even more unexpectedly, (ii) appeared to have induced an immune memory response against subsequent tumor re-challenge.

In the field of cancer treatment, it is increasingly obvious that “one size fits all” solutions are scarce. The inventors therefore explored whether the effect of SLC4A4 inhibition on potentiation of immune checkpoint inhibitor response is limited to pancreatic cancer. Surprisingly, certainly in view of the prior art not allowing to expect any success as to whether SLC4A4 inhibition would be able to modulate a subject's immune response to any cancer, it was demonstrated that this potentiation effect extended to other cancers as well as to other immune checkpoint inhibitors. Indeed, the non-response or poor response of glioblastoma and lung cancer to anti-PDL1 was potentiated by SLC4A4 inhibition, and furthermore the non-response or poor response of lung cancer to anti-CTLA-4 was potentiated by SLC4A4 inhibition. The effect of SLC4A4 inhibition on potentiation of immune checkpoint inhibitor response thus is more broadly applicable in not being limited to a single cancer type, and is not being limited to a single immune checkpoint inhibitor. Further work on other combinations and on colorectal cancer is ongoing.

Therefore, in a first aspect, the invention relates to an inhibitor of Solute Carrier Family 4 member 4 (SLC4A4) for use in treating or inhibiting cancer or a tumor, or for use in inhibiting progression, relapse or metastasis of cancer or of a tumor, wherein the cancer or tumor is poorly responding to or is resistant to immunotherapy, or is poorly responding to or is resistant to treatment or therapy comprising immunotherapy. Alternatively, the invention relates to use of an inhibitor of Solute Carrier Family 4 member 4 (SLC4A4) in the manufacture of a medicine or medicament for treating or inhibiting cancer or a tumor, or for use in inhibiting progression, relapse or metastasis of cancer or a tumor, wherein the cancer or tumor is poorly responding to or is resistant to immunotherapy, or is poorly responding to or is resistant to treatment or therapy comprising immunotherapy. Further alternatively, the invention relates to methods of treating or inhibiting cancer or a tumor, or of inhibiting progression, relapse or metastasis of cancer or a tumor, in a subject, individual or patient (in particular a mammalian subject or mammal, such as a human subject or human), such methods comprising administering an inhibitor of SLC4A4 to the subject or individual, and wherein the cancer or tumor is poorly responding to or is resistant to immunotherapy, or is poorly responding to or is resistant to treatment or therapy comprising immunotherapy. The administration of the SLC4A4 inhibitor, such as a therapeutically effective amount of the SLC4A4 inhibitor, to the subject, individual or patient results in the treatment or inhibition of cancer or tumor growth, or in inhibition of the progression, relapse or metastasis of cancer or tumor growth.

In particular, the immunotherapy is a treatment or therapy with an immune checkpoint inhibitor, or a treatment or therapy comprising an immune checkpoint inhibitor. Poor response or resistance to immunotherapy (such as checkpoint inhibitor therapy) is herewith understood as either non-response (NR) or partial response (PR) to the immunotherapy (such as immune checkpoint inhibitor therapy), in particular to a therapy consisting of administration of immunotherapy only, or in particular to a therapy comprising administration of an immunotherapeutic compound or agent. When such therapy is comprising administration of an immunotherapeutic compound or agent, it is in particular not comprising, or is excluding, administration of an inhibitor of SLC4A4. The poor response, resistance, non-response or partial response in particular may be based on clinical experience or observation and/or may be based on analysis of biomarkers that are predictive or prognostic for efficacy of immune therapy (such as immune checkpoint inhibitor treatment or therapy). Such biomarkers may be analyzed in tumor tissue (e.g. tumor biopsy) or in a liquid biopsy taken e.g. from the patient's circulation (e.g. cfDNA, ctDNA, circulating cancer cells, exosomes, serum proteins . . . ).

Cancer immunotherapy has provided patients with a promising treatment option. Therapeutic regimens such as adoptive T cell transfer (ACT), cancer vaccines and immune checkpoint inhibitors (e.g. anti-PD-1, anti-PDL1 or anti-CTLA-4 antibodies), harness the ability of the immune system to recognize and reject the tumor (Smyth et al. 2015, Nat Rev Clin Oncol 13:143-158). However, despite high response rates, with prolonged survival in a subset of melanoma (e.g. Schadendorf et al. 2015, J Clin Oncol 33:1889-189), lung (e.g. Borghaei et al. 2015, N Engl J Med 373:1627-1639), and renal cancer patients (e.g. Motzer et al. 2015, N Engl J Med 373:1803-1813), for several other tumors such as mismatch repair (MMR)-proficient colorectal cancer (CRC) (e.g. Le et al. 2015, N Engl J Med 372: 1509-2520) and pancreatic ductal adenocarcinoma (PDAC) (e.g. Sarantis et al. 2020, World J Gastrointest Oncol 12: 173-181) immunotherapy fails to show any clinical benefit.

The invention in a further aspect relates to an inhibitor of Solute Carrier Family 4 member 4 (SLC4A4) for use in treating or inhibiting pancreatic cancer or for use in inhibiting progression, relapse or metastasis of pancreatic cancer. Alternatively, the invention relates to use of an inhibitor of Solute Carrier Family 4 member 4 (SLC4A4) in the manufacture of a medicine or medicament for treating or inhibiting pancreatic cancer or for use in inhibiting progression, relapse or metastasis of pancreatic cancer. Further alternatively, the invention relates to methods of treating or inhibiting pancreatic cancer or of inhibiting progression, relapse or metastasis of pancreatic cancer, in a subject, individual or patient (in particular a mammalian subject or mammal, such as a human subject or human), such methods comprising administering an inhibitor of SLC4A4 to the subject or individual. The administration of the SLC4A4 inhibitor, such as a therapeutically effective amount of the SLC4A4 inhibitor, to the subject, individual or patient results in the treatment or inhibition of pancreatic cancer or tumor growth, or in inhibition of the progression, relapse or metastasis of pancreatic cancer or tumor growth.

Pancreatic adenocarcinoma (PDAC) is one of the most aggressive and lethal cancer types. The projected doubling of the incidence of PDAC by 2030 would make it the second most common cause of cancer-related death, following lung cancer. Tumors develop rapidly, invading surrounding tissues with the consequence that fewer than 20% of the patients are eligible for resection at the moment of diagnosis (Pereira et al. 2020, The Lancet Gastroentrol Hepatol 5:698-710). Most of the therapies including the recent immunotherapeutic approaches are not effective (Royal et al. 2010, J Immunother 33:828-833), and the majority of those patients that do proceed with surgery will ultimately relapse (Strobel et al. 2017, Ann Surg 265:565-573; Kamisawa et al. 2016, The Lancet 388:73-85). Therefore, there is an urgent need for treatments applicable to the vast majority of patients with unresectable tumors or that prevent post-surgical relapse (Neoptolemos et al. 2017, The Lancet 389:1011-1024). PDACs are characterized by a dense desmoplastic stroma that impedes oxygen and nutrient diffusion from the blood stream and contributes to a strong hypoxic and acidic tumor microenvironment (TME) (Gajewski et al. 2013, Nat Immunol 14:1014-1022; Whatcott et al. 2015, Clin Cancer Res 21:3561-3568) . In this harsh TME, cytotoxic T cells struggle to enter or to work efficiently (Joyce et al. 2015, Science 348, 74-80), also because pancreatic cancer cells are poorly recognized by the immune system due to the downregulation of the major histocompatibility complex class I (Yamamoto et al. 2020, Nature 581:100-105). Preclinical and clinical efforts have been pursued to make pancreatic tumors more immunogenic. These efforts encompass the combination of immune checkpoint inhibitors with pharmacological strategies targeting immunosuppressive fibrobasts, myeloid cells, or regulatory T cells, as well as cancer vaccines (e.g. GVAX) genetically modified to release immune stimulatory cytokines (e.g. Jaffee et al. 2001, J Clin Oncol 19:145-156; Lutz et al. 2011, Ann Surg 253:328-335; Özdemir et al. 2014, Cancer Cell 25:719-734; Rhim et al. 2014, Cancer Cell 25:735-747; Elyada et al. 2019, Cancer Discov 9:1102-1123; Mantovani et al. 2017, Nat Rev Clin Oncol 14:399-416; Huelsken & Hanahan 2018, Cell 172:643-644; Zhu et al. 2017, Immunity 47:323-338). Nevertheless, none of these approaches has reached the desired effects so far.

In yet a further aspect, the invention relates to an inhibitor of Solute Carrier Family 4 member 4 (SLC4A4) for use in treating or inhibiting cancer or for use in inhibiting progression, relapse or metastasis of cancer, in combination with immunotherapy; or wherein the treatment or inhibition further comprises therapy with or administration of an immunotherapy/immunotherapeutic compound or agent; or wherein the treatment or inhibition is combined with a therapy comprising an immunotherapy or with administration of an immunotherapy/immunotherapeutic compound or agent (to the subject, individual or patient having the cancer or tumor).

Alternatively, the invention relates to use of an inhibitor of SLC4A4 in the manufacture of a medicament for use in combination with (administration of) an immunotherapy/immunotherapeutic compound or agent for treating or inhibiting cancer or for inhibiting progression, relapse or metastasis of cancer (in a subject, individual or patient having the cancer or tumor); or wherein the treatment or inhibition further comprises therapy with or administration of an immunotherapy/immunotherapeutic compound or agent; or wherein the treatment or inhibition is combined with a therapy comprising an immunotherapy or with administration of an immunotherapy/immunotherapeutic compound or agent (to the subject, individual or patient having the cancer or tumor).

Alternatively, the invention relates to use of an inhibitor of SLC4A4 in the manufacture of a medicament for treating or inhibiting cancer or for inhibiting progression, relapse or metastasis of cancer (in a subject, individual or patient having the cancer) in combination with an immunotherapy (for treating or inhibiting cancer or for inhibiting progression, relapse or metastasis of cancer); or in combination with administering an immunotherapy/immunotherapeutic compound or agent to the subject, individual or patient; or wherein the treatment or inhibition further comprises therapy with or administration of an immunotherapy/immunotherapeutic compound or agent; or wherein the treatment or inhibition is combined with a therapy comprising an immunotherapy or with administration of an immunotherapy/immunotherapeutic compound or agent (to the subject, individual or patient having the cancer or tumor).

In an alternative aspect, the invention relates to an immunotherapy for use in treating or inhibiting cancer or for use in inhibiting progression, relapse or metastasis of cancer, in combination with (administration of) an inhibitor of SLC4A4; or wherein the treatment or inhibition further comprises therapy with or administration of an inhibitor of SLC4A4; or wherein the treatment or inhibition is combined with a therapy comprising an inhibitor of SLC4A4 or with administration of an inhibitor of SLC4A4 (to the subject, individual or patient having the cancer or tumor).

Alternatively, the invention relates to use of an immunotherapeutic compound or agent in the manufacture of a medicament for treating or inhibiting cancer or for inhibiting progression, relapse or metastasis of cancer (in a subject, individual or patient having the cancer) in combination with an inhibitor of SLC4A4 (for treating or inhibiting cancer or for inhibiting progression, relapse or metastasis of cancer); or in combination with administering an inhibitor of SLC4A4 to the subject, individual or patient; or wherein the treatment or inhibition further comprises therapy with or administration of an inhibitor of SLC4A4; or wherein the treatment or inhibition is combined with a therapy comprising an inhibitor of SLC4A4 or with administration of an inhibitor of SLC4A4 (to the subject, individual or patient having the cancer or tumor).

In another alternative aspect, the invention relates to an inhibitor of SLC4A4 and an immunotherapy for use in treating or inhibiting cancer or for use in inhibiting progression, relapse or metastasis of cancer. Alternatively, the invention relates to use of an inhibitor of SLC4A4 and (use of) an immunotherapy/immunotherapeutic compound or agent in the manufacture of a medicament for use in treating or inhibiting cancer or for inhibiting progression, relapse or metastasis of cancer (in a subject, individual or patient having the cancer).

A further aspect of the invention relates to a method for treating or inhibiting cancer, or a method for inhibiting progression, relapse or metastasis of cancer, in a subject, individual or patient (in particular a mammalian subject or mammal, such as a human subject or human), the methods comprising administering an inhibitor of SLC4A4 and administering an immunotherapy/immunotherapeutic compound or agent to the subject, individual or patient. By administering the inhibitor of SLC4A4 and the immunotherapy/immunotherapeutic compound or agent, the cancer is treated or inhibited, or the progression, relapse or metastasis of the cancer is inhibited. In particular, an effective amount of the inhibitor of SLC4A4 and of the immunotherapy/immunotherapeutic compound or agent is administered to the subject, individual or patient; or an effective amount of a combination (in any way) of the inhibitor of SLC4A4 and of the immunotherapy/immunotherapeutic compound or agent is administered to the subject, individual or patient.

In any of the above, the inhibitor of SLC4A4 may in particular be a specific inhibitor of SLC4A4 or a selective inhibitor of SLC4A4.

In any of the above, the immunotherapy in one embodiment is a treatment or therapy with an immune checkpoint inhibitor or a treatment or therapy comprising an immune checkpoint inhibitor (the immunotherapeutic compound or agent in this case thus is an immune checkpoint inhibitor). In a further embodiment, the immunotherapy is a treatment or therapy with two immune checkpoint inhibitors or a treatment or therapy comprising two immune checkpoint inhibitors (the immunotherapeutic compound or agent in this case thus is a combination of two immune checkpoint inhibitors). In a particular embodiment, said two immune checkpoint inhibitors are then chosen such that each of the two inhibitors is inhibiting a different immune checkpoint protein or a different immune checkpoint protein-ligand interaction.

In any of the above aspects and embodiments, the combination is in particular a combination in any way or in any appropriate way (explained in more detail hereinafter).

In any of the above aspects and embodiments, the inhibitor of SLC4A4 may be a genetic inhibitor of SLC4A4, a genetic inhibitor specific to SLC4A4, a pharmacological inhibitor of SLC4A4, or a pharmacological inhibitor specific to SLC4A4 (specificity and selectivity of inhibition are explained in more detail hereinafter).

In particular, a genetic inhibitor of SLC4A4 may be an inhibitory oligonucleotide specifically targeting SLC4A4. Such inhibitory oligonucleotide specifically targeting SLC4A4 may be selected from (the group consisting of) an antisense oligomer, a siRNA, a shRNA, a gapmer, and the likes.

In particular, a pharmacological inhibitor of SLC4A4 may be selected from (the group consisting of) a polypeptide comprising an immunoglobulin variable domain, a monoclonal antibody or a fragment thereof, an alpha-body, a nanobody, an intrabody, an aptamer, a DARPin, an affibody, an affitin, an anticalin, a monobody, a bicyclic peptide, a PROTAC, a LYTAC. Immunoglobulin variable domains, monoclonal antibodies or fragments thereof, alpha-bodies, nanobodies, intrabodies, aptamers, DARPins, affibodies, affitins, anticalins, monobodies, and bicyclic peptides can be screened for inhibiting SLC4A4 activity in largely similar ways. Identification of an inhibitor of SLC4A4 in one of these classes thus plausibly supports identification, without undue burden, of an inhibitor of SLC4A4 in any other class; compounds of all these classes are furthermore known to come with high specificity or selectivity to their target. The group of pharmacological inhibitors of SLC4A4 can be extended with DNA nucleases specifically knocking out or disrupting SLC4A4, and RNases specifically targeting SLC4A4. Such DNA nuclease specifically knocking out or disrupting SLC4A4 may be selected from (the group consisting of) a ZFN, a TALEN, a CRISPR-Cas, and a meganuclease. Such RNase specifically targeting SLC4A4 may be selected from (the group consisting of) a ribozyme and a CRISPR-C2c2.

In any of the above aspects and embodiments, the inhibitors of the two different immune checkpoint protein-ligand interactions are e.g. a PD1 inhibitor and a CTLA4 inhibitor.

In any of the above aspects and embodiments, the two different immune checkpoint-ligand interactions are e.g. two selected from (the group consisting of) PD1 with ligand PDL1, PD1 with ligand PDL2, CTLA4 with ligand B7-1, CTLA4 with ligand B7-2.

In any of the above aspects and embodiments, the tumor or cancer may in particular embodiments be a pancreatic tumor or cancer, a lung tumor or cancer, glioblastoma, or a colorectal tumor or cancer. In any of the above aspects and embodiments, the tumor or cancer in particular is poorly responding to, resistant to, or refractory to immunotherapy or to therapy comprising an immunotherapeutic compound or agent.

The term “antagonist” or “inhibitor” of a target as used interchangeable herein refers to inhibitors of function or to inhibitors of expression of a target of interest. Antagonists or inhibitors of a target may also be compounds binding to a target (e.g. tumor) cell and causing its killing; examples of such antagonists include e.g. antibody-(cytotoxic) drug-conjugates or antibodies capable of causing ADCC. Interchangeable alternatives for “antagonist” include inhibitor, repressor, suppressor, inactivator, and blocker. An “antagonist” thus refers to a molecule that decreases, blocks, inhibits, abrogates, or interferes with target expression, activation, function, or activity.

Downregulating of expression of a gene encoding a target is feasible through gene therapy (e.g., by administering siRNA, shRNA or antisense oligonucleotides to the target gene). Biopharmaceutical and gene therapeutic antagonists include such entities as antisense oligonucleotides, gapmers, siRNA, shRNA, zinc-finger nucleases, meganucleases, TAL effector nucleases, CRISPR-Cas effectors, monoclonal antibodies or fragments thereof, alpha-bodies, nanobodies, intrabodies, aptamers, DARPins, affibodies, affitins, anticalins, monobodies, PROTACs, LYTACs, etc. (general description of these compounds included hereinafter).

Inactivation of a process as envisaged in the current invention refers to different possible levels of inactivation, e.g., at least 10%, at least 20%, at least 30%, at least 40%, at least 50%, at least 60%, at least 70%, at least 80%, at least 90%, at least 95%, or even 100% or more if inactivation (compared to a normal situation). The nature of the inactivating compound is not vital/essential to the invention as long as the process envisaged is inactivated such as to treat or inhibit cancer or tumor growth or such as to inhibit progression, relapse or metastasis of cancer or tumor growth.

### Inhibition of a Target of Interest

Downregulating expression of a gene encoding a target is feasible through agents include entities such as antisense oligonucleotides, gapmers, siRNA, shRNA, zinc-finger nucleases, meganucleases, Argonaute, TAL effector nucleases, CRISPR-Cas effectors, and nucleic acid aptamers. In particular, any of these agents is specifically, selectively, or exclusively acting on or antagonizing the target of interest; or any of these agents is designed for specifically, selectively, or exclusively acting on or antagonizing the target of interest.

One process of modulating/downregulating expression of a gene/target gene of interest relies on antisense oligonucleotides (ASOs), or variants thereof such as gapmers. An antisense oligonucleotide (ASO) is a short strand of nucleotides and/or nucleotide analogues that hybridizes with the complementary mRNA in a sequence-specific or -selective manner. Formation of the ASO-mRNA complex ultimately results in downregulation of target protein expression (Chan et al. 2006, Clin Exp Pharmacol Physiol 33:533-540; this reference also describes some of the software available for assisting in design of ASOs). Modifications to ASOs can be introduced at one or more levels: phosphate linkage modification (e.g. introduction of one or more of phosphodiester, phosphoramidate or phosphorothioate bonds), sugar modification (e.g. introduction of one or more of LNA (locked nucleic acids), 2′-O-methyl, 2′-O-methoxy-ethyl, 2′-fluoro, S-constrained ethyl or tricyclo-DNA and/or non-ribose modifications (e.g. introduction of one or more of phosphorodiamidate morpholinos or peptide nucleic acids). The introduction of 2′-modifications has been shown to enhance safety and pharmacologic properties of antisense oligonucleotides. Antisense strategies relying on degradation of mRNA by RNase H requires the presence of nucleotides with a free 2′-oxygen, i.e. not all nucleotides in the antisense molecule should be 2′-modified. The gapmer strategy has been developed to this end. A gapmer antisense oligonucleotide consists of a central DNA region (usually a minimum of 7 or 8 nucleotides) with (usually 2 or 3) 2′-modified nucleosides flanking both ends of the central DNA region. This is sufficient for the protection against exonucleases while allowing RNAseH to act on the (2′-modification free) gap region. Antidote strategies are available as demonstrated by administration of an oligonucleotide fully complementary to the antisense oligonucleotide (Crosby et al. 2015, Nucleic Acid Ther 25:297-305).

Another process to modulate expression of a gene/target gene of interest is based on the natural process of RNA interference. It relies on double-stranded RNA (dsRNA) that is cut by an enzyme called Dicer, resulting in double stranded small interfering RNA (siRNA) molecules which are 20-25 nucleotides long. siRNA then binds to the cellular RNA-Induced Silencing Complex (RISC) separating the two strands into the passenger and guide strand. While the passenger strand is degraded, RISC is cleaving mRNA specifically or selectively at a site instructed by the guide strand. Destruction of the mRNA prevents production of the protein of interest and the gene is ‘silenced’. siRNAs are dsRNAs with 2 nt 3′ end overhangs whereas shRNAs are dsRNAs that contains a loop structure that is processed to siRNA. shRNAs are introduced into the nuclei of target cells using a vector (e.g. bacterial or viral) that optionally can stably integrate into the genome. Apart from checking for lack of cross-reactivity with non-target genes, manufacturers of RNAi products provide guidelines for designing siRNA/shRNA. siRNA sequences between 19-29 nt are generally the most effective. Sequences longer than 30 nt can result in nonspecific silencing. Ideal sites to target include AA dinucleotides and the 19 nt 3′ of them in the target mRNA sequence. Typically, siRNAs with 3′ dUdU or dTdT dinucleotide overhangs are more effective. Other dinucleotide overhangs could maintain activity but GG overhangs should be avoided. Also to be avoided are siRNA designs with a 4-6 poly(T) tract (acting as a termination signal for RNA pol III), and the G/C content is advised to be between 35-55%. shRNAs should comprise sense and antisense sequences (advised to each be 19-21 nt in length) separated by loop structure, and a 3′ AAAA overhang. Effective loop structures are suggested to be 3-9 nt in length. It is suggested to follow the sense-loop-antisense order in designing the shRNA cassette and to avoid 5′ overhangs in the shRNA construct. shRNAs are usually transcribed from vectors, e.g. driven by the Pol III U6 promoter or H1 promoter. Vectors allow for inducible shRNA expression, e.g. relying on the Tet-on and Tet-off inducible systems commercially available, or on a modified U6 promoter that is induced by the insect hormone ecdysone. A Cre-Lox recombination system has been used to achieve controlled expression in mice. Synthetic shRNAs can be chemically modified to affect their activity and stability. Plasmid DNA or dsRNA can be delivered to a cell by means of transfection (lipid transfection, cationic polymer-based nanoparticles, lipid or cell-penetrating peptide conjugation) or electroporation. Vectors include viral vectors such as lentiviral, retroviral, adenoviral and adeno-associated viral vectors.

Ribozymes (ribonucleic acid enzymes) are another type of molecules that can be used to modulate expression of a gene/target gene of interest. They are RNA molecules capable of catalyzing specific biochemical reactions, in the current context capable of targeted cleavage of nucleotide sequences, in particular targeted cleavage of a RNA/RNA target of interest. Examples of ribozymes include the hammerhead ribozyme, the Varkud Satellite ribozyme, Leadzyme and the hairpin ribozyme.

Besides the use of the inhibitory RNA technology, modulation of expression of a gene of interest can be achieved at DNA level such as by gene therapy to knock-out, knock-down or disrupt the target gene/gene of interest. As used herein, a “gene knock-out” can be a gene knockdown or the gene can be knocked out, knocked down, disrupted or modified by a mutation such as, a point mutation, an insertion, a deletion, a frameshift, or a missense mutation by techniques such as described hereafter, including, but not limited to, retroviral gene transfer. One way in which genes can be knocked out, knocked down, disrupted or modified is by the use of zinc finger nucleases. Zinc-finger nucleases (ZFNs) are artificial restriction enzymes generated by fusing a zinc finger DNA-binding domain to a DNA-cleavage domain. Zinc finger domains can be engineered to target a desired DNA sequence/DNA sequence of interest, which enable zinc-finger nucleases to target unique sequence within a complex genome. By taking advantage of the endogenous DNA repair machinery, these reagents can be used to precisely alter the genomes of higher organisms.

Other technologies for genome customization that can be used to specifically or selectively knock out, knock down or disrupt a gene/gene of interest are meganucleases and TAL effector nucleases (TALENs, Cellectis bioresearch). A TALEN® is composed of a TALE DNA binding domain for sequence-specific or sequence-selective recognition fused to the catalytic domain of an endonuclease that introduces double strand breaks (DSB). The DNA binding domain of a TALEN® is capable of targeting with high precision a large recognition site (for instance 17 bp). Meganucleases are sequence-specific or sequence-selective endonucleases, naturally occurring “DNA scissors”, originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes) or DNA sequences of interest. Another recent genome editing technology is the CRISPR/Cas system, which can be used to achieve RNA-guided genome engineering (including knock-out, knock-down or disruption of a gene of interest). CRISPR interference is a genetic technique which allows for sequence-specific or sequence-selective control of expression of a gene of interest in prokaryotic and eukaryotic cells. It is based on the bacterial immune system-derived CRISPR (clustered regularly interspaced palindromic repeats) pathway. Recently, it was demonstrated that the CRISPR-Cas editing system can also be used to target RNA. It has been shown that the Class 2 type VI-A CRISPR-Cas effector C2c2 (Cas13a; CRISPR-Cas13a or CRISPR-C2c2) can be programmed to cleave single stranded RNA targets carrying complementary protospacers (Abudayyeh et al. 2016 Science353/science.aaf5573). C2c2 is a single-effector endoRNase mediating ssRNA cleavage once it has been guided by a single crRNA guide toward a target RNA/RNA of interest.

Methods for administering nucleic acids include methods applying non-viral (DNA or RNA) or viral nucleic acids (DNA or RNA viral vectors). Methods for non-viral gene therapy include the injection of naked DNA (circular or linear), electroporation, the gene gun, sonoporation, magnetofection, the use of oligonucleotides, lipoplexes (e.g. complexes of nucleic acid with DOTAP or DOPE or combinations thereof, complexes with other cationic lipids), dendrimers, viral-like particles, inorganic nanoparticles, hydrodynamic delivery, photochemical internalization (Berg et al. 2010, Methods Mol Biol 635:133-145) or combinations thereof.

Many different vectors have been used in human nucleic acid therapy trials and a listing can be found on http://www.abedia.com/wiley/vectors.php. Currently the major groups are adenovirus or adeno-associated virus vectors (in about 21% and 7% of the clinical trials), retrovirus vectors (about 19% of clinical trials), naked or plasmid DNA (about 17% of clinical trials), and lentivirus vectors (about 6% of clinical trials). Combinations are also possible, e.g. naked or plasmid DNA combined with adenovirus, or RNA combined with naked or plasmid DNA to list just a few. Other viruses (e.g. alphaviruses, vaccinia viruses such as vaccinia virus Ankara) are used in nucleic acid therapy and are not excluded in the context of the current invention.

Administration may be aided by specific formulation of the nucleic acid e.g. in liposomes (lipoplexes) or polymersomes (synthetic variants of liposomes), as polyplexes (nucleic acid complexed with polymers), carried on dendrimers, in inorganic (nano)particles (e.g. containing iron oxide in case of magnetofection), or combined with a cell penetrating peptide (CPP) to increase cellular uptake. Organ- or cellular-targeting strategies may also be applied to the nucleic acid (nucleic acid combined with organ- or cell-targeting moiety); these include passive targeting (mostly achieved by adapted formulation) or active targeting (e.g. by coupling a nucleic acid-comprising nanoparticle with any compound (e.g. an aptamer or antibody or antigen binding molecule) binding to a target organ- or cell-specific antigen) (e.g. Steichen et al. 2013, Eur J Pharm Sci 48:416-427).

CPPs enable translocation of the drug of interest coupled to them across the plasma membrane. CPPs are alternatively termed Protein Transduction Domains (TPDs), usually comprise 30 or less (e.g. 5 to 30, or 5 to 20) amino acids, and usually are rich in basic residues, and are derived from naturally occurring CPPs (usually longer than 20 amino acids), or are the result of modelling or design. A non-limiting selection of CPPs includes the TAT peptide (derived from HIV-1 Tat protein), penetratin (derived from Drosophila Antennapedia—Antp), pVEC (derived from murine vascular endothelial cadherin), signal-sequence based peptides or membrane translocating sequences, model amphipathic peptide (MAP), transportan, MPG, polyarginines; more information on these peptides can be found in Torchilin 2008 (Adv Drug Deliv Rev 60:548-558) and references cited therein. CPPs can be coupled to carriers such as nanoparticles, liposomes, micelles, or generally any hydrophobic particle. Coupling can be by absorption or chemical bonding, such as via a spacer between the CPP and the carrier. To increase target specificity or target selectivity, an antibody binding to a target-specific antigen can further be coupled to the carrier (Torchilin 2008, Adv Drug Deliv Rev 60:548-558). CPPs have already been used to deliver payloads as diverse as plasmid DNA, oligonucleotides, siRNA, peptide nucleic acids (PNA), proteins and peptides, small molecules and nanoparticles inside the cell (Stalmans et al. 2013, PloS One 8:e71752).

Any other modification of the DNA or RNA to enhance efficacy of nucleic acid therapy is likewise envisaged to be useful in the context of the applications of the genetic inhibitor as outlined herein. The enhanced efficacy can reside in enhanced expression, enhanced delivery properties, enhanced stability and the like. The applications of the genetic inhibitor as outlined herein may thus rely on using a modified nucleic acid as described above. Further modifications of the nucleic acid may include those suppressing inflammatory responses (hypoinflammatory nucleic acids).

Pharmacological inhibition of a target of interest in general occurs by means of an agent inhibiting at least one of the biological activities (if more than one is known) of a target protein of interest. In particular, such pharmacological inhibitor is binding, such as specifically, selectively and/or exclusively binding to a target protein or protein of interest, or is specifically, selectively and/or exclusively inhibiting the targeted biological activity of the a target protein of interest.

Such binding may occur with high affinity although this is not an absolute requirement. The pharmacological inhibitor of a target protein or protein of interest may for instance have a binding affinity (dissociation constant) to (one of) its target of about 1000 nM or less, a binding affinity of about 100 nM or less, a binding affinity of about 50 nM or less, a binding affinity of about 10 nM or less, or a binding affinity of about 1 nM or less. Cross-reactivity of a pharmacological inhibitor with more than one protein is possible; for clinical development it can e.g. be desired to be able to test a pharmacological inhibitor in a suitable in vitro model or in vivo animal model before starting clinical testing with the same pharmacological inhibitor in a human population, which may require the pharmacological inhibitor to cross-react with the animal (or other non-human) target protein and with the orthologous human target protein (orthologous proteins are homologous proteins separated by a speciation event).

Specificity or selectivity of binding refers to the situation in which a pharmacological inhibitor is, at a certain concentration (sufficient to inhibit the target protein or protein of interest) binding to the target protein with higher affinity (e.g. at least 2-fold, 5-fold, or at least 10-fold higher affinity, e.g. at least 20-, 50- or 100-fold or more higher affinity) than the affinity with which it is possibly (if at all) binding to other proteins (proteins not of interest). Such specificity or selectivity of binding is in particular determined within the setting of the target subject (e.g. human patient, or animal model) and thus can encompass/does not exclude binding to (at least one) orthologous target proteins. Exclusivity of binding refers to the situation in which a pharmacological inhibitor is binding only to the target protein of interest (and possibly to (at least one) orthologous target protein).

Alternatively, the pharmacological inhibitor may exert the desired level of inhibition of the targeted biological activity or biological activity of interest of a target protein or protein of interest with an IC50 of 1000 nM or less, with an IC50 of 500 nM or less, with an IC50 of 100 nM or less, with an IC50 of 50 nM or less, with an IC50 of 10 nM or less, or with an IC50 of 1 nM or less.

Cross-inhibition by a pharmacological inhibitor of more than one protein is possible; for clinical development it can e.g. be desired to be able to test a pharmacological inhibitor in a suitable in vitro model or in vivo animal model before starting clinical testing with the same pharmacological inhibitor in a human population, which may require the pharmacological inhibitor to cross-inhibit the animal (or other non-human) target protein and the orthologous human target protein.

Specificity or selectivity of inhibition refers to the situation in which a pharmacological inhibitor is, at a certain concentration (sufficient to inhibit the target protein or protein of interest) inhibiting the target protein with higher efficacy (e.g. with an at least 2-fold, 5-fold, or 10-fold lower IC50, e.g. at least 20-,50- or 100-fold or more lower IC50) than the efficacy with which it is possibly (if at all) inhibiting other proteins (proteins not of interest). Such specificity or selectivity of inhibition is in particular determined within the setting of the target subject (e.g. human patient, or animal model) and thus can encompass/does not exclude inhibition of (at least one) orthologous target proteins. Exclusivity of inhibition refers to the situation in which a pharmacological inhibitor is inhibiting only the target protein of interest (or (at least one) orthologous target protein).

Specificity or selectivity of inhibition may refer to inhibition of a single biological activity of a protein of interest (and possibly of (at least one) orthologue) if the protein of interest is known to have more than one biological activity; or may refer to inhibition of the protein of interest (and possibly of (at least one) orthologue) as such, independent of it possibly having multiple biological activities.

Exclusivity of inhibition refers to the situation in which a pharmacological inhibitor is inhibiting only a single biological activity of a protein of interest (and possibly of (at least one) orthologue) if the protein of interest is known to have more than one biological activity; or may refer to inhibition of only the protein of interest (and possibly of (at least one) orthologue) as such, independent of it possibly having multiple biological activities.

In general, the agent inhibiting a target protein or protein of interest is a polypeptide, a polypeptidic agent, an aptamer, or a combination of any of the foregoing. Examples of such pharmacologic inhibitors, all specifically, selectively and/or exclusively binding to and/or inhibiting the target protein of interest include immunoglobulin variable domains, antibodies (in particular monoclonal antibodies) or a fragment thereof, alpha-bodies, nanobodies, intrabodies, aptamers, DARPins, affibodies, affitins, anticalins, monobodies, and bicyclic peptides.

The term “antibody” as used herein, refers to an immunoglobulin (Ig) molecule, which specifically or selectively binds with an antigen. Antibodies can be intact immunoglobulins derived from natural sources or from recombinant sources and can be immunoreactive portions of intact immunoglobulins. Antibodies are typically tetramers of immunoglobulin molecules. The term “immunoglobulin domain” as used herein refers to a globular region of an antibody chain (such as e.g., a chain of a conventional 4-chain antibody or a chain of a heavy chain antibody), or to a polypeptide that essentially consists of such a globular region/immunoglobulin domain. Immunoglobulin domains are characterized in that they retain the immunoglobulin fold characteristic of antibody molecules, which consists of a two-layer sandwich of about seven antiparallel β-strands arranged in two β-sheets, optionally stabilized by a conserved disulphide bond.

The specificity or selectivity of an antibody/immunoglobulin/immunoglobulin domain/immunoglobulin variable domain (IVD) for an antigen is defined by the composition of the antigen-binding domains in the antibody/immunoglobulin/IVD (usually one or more of the CDRs, the particular amino acids of the antibody/immunoglobulin/IVD interacting with the antigen, and forming the paratope or antigen-binding site) and the composition of the antigen (the parts of the antigen interacting with the antibody/immunoglobulin/IVD and forming the epitope or antibody binding site). Specificity or selectivity of binding is understood to refer to a binding between an antibody/immunoglobulin/IVD with a single target molecule or with a limited number of target molecules that (happen to) share an epitope recognized by the antibody/immunoglobulin/IVD.

Affinity of an antibody/immunoglobulin/IVD for its target is a measure for the strength of interaction between an epitope on the target (antigen) and an epitope/antigen binding site in the antibody/immunoglobulin/IVD. It can be defined as:

$${K}_{A} = \frac{[ Ab - Ag ]}{[ Ab ] [ Ag ]}$$

Wherein KA is the affinity constant, [Ab] is the molar concentration of unoccupied binding sites on the antibody/immunoglobulin/IVD, [Ag] is the molar concentration of unoccupied binding sites on the antigen, and [Ab-Ag] is the molar concentration of the antibody-antigen complex. Avidity provides information on the overall strength of an antibody/immunoglobulin/IVD-antigen complex, and generally depends on the above-described affinity, the valency of antibody/immunoglobulin/IVD and of antigen, and the structural interaction of the binding partners.

The term “immunoglobulin variable domain” (abbreviated as “IVD”) as used herein means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) (IVDs) that confer specificity or selectivity to an antibody for the antigen by carrying the antigen-binding site. Methods for delineating/confining a CDR in an antibody/immunoglobulin/immunoglobulin domain/IVD have been described in the art and include the Kabat, Chothia, IMTG, Martin, Gelfand, and Honneger systems (see Dondelinger et al. 2018, Front Immunol 9:2278).

The term “immunoglobulin single variable domain” (abbreviated as “ISVD”), equivalent to the term “single variable domain”, defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associated) immunoglobulin domains such as light and heavy chain variable domains, i.e., by a VH-VL pair of immunoglobulin domains, which jointly bind to an epitope of the respective antigen. In contrast, immunoglobulin single variable domains are capable of specifically or selectively binding to an epitope of the antigen without pairing with an additional immunoglobulin variable domain. The binding site of an immunoglobulin single variable domain is formed by a single VH/VHH or VL domain. Hence, the antigen binding site of an immunoglobulin single variable domain is formed by no more than three CDRs. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit). In one embodiment of the invention, the immunoglobulin single variable domains are heavy chain variable domain sequences (e.g., a VH-sequence); more specifically, the immunoglobulin single variable domains can be heavy chain variable domain sequences that are derived from a conventional four-chain antibody or heavy chain variable domain sequences that are derived from a heavy chain antibody. For example, the immunoglobulin single variable domain may be a (single) domain antibody (or an amino acid sequence that is suitable for use as a (single) domain antibody), a “dAb” or dAb (or an amino acid sequence that is suitable for use as a dAb) or a Nanobody® (as defined herein, and including but not limited to a VHH); other single variable domains, or any suitable fragment of any one thereof. In particular, the immunoglobulin single variable domain may be a Nanobody® (as defined herein) or a suitable fragment thereof. Note: Nanobody®, Nanobodies® and Nanoclone® are registered trademarks of Ablynx (now part of Sanofi). For a general description of Nanobodies®, reference is made to the further description below, as well as to e.g. WO2008/020079. “VHH domains”, also known as VHHs, VHH domains, VHH antibody fragments, and VHH antibodies, have originally been described as the antigen binding immunoglobulin (variable) domain of “heavy chain antibodies” (i.e., of “antibodies devoid of light chains”; Hamers-Casterman et al. 1993, Nature 363:446-448). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobody®, reference is made to the review article by Muyldermans 2001 (Rev Mol Biotechnol 74:277-302), as well as to the following patent applications, which are mentioned as general background art: WO 94/04678, WO 95/04079, WO 96/34103; WO 94/25591, WO 99/37681, WO 00/40968, WO 00/43507, WO 00/65057, WO 01/40310, WO 01/44301, EP 1134231 and WO 02/48193; WO 97/49805, WO 01/21817, WO 03/035694, WO 03/054016 and WO 03/055527; WO 03/050531; WO 01/90190; WO 03/025020; WO 04/041867, WO 04/041862, WO 04/041865, WO 04/041863, WO 04/062551, WO 05/044858, WO 06/40153, WO 06/079372, WO 06/122786, WO 06/122787 and WO 06/122825. As described in these references, Nanobody® (in particular VHH sequences and partially humanized Nanobody®) can in particular be characterized by the presence of one or more “hallmark residues” in one or more of the framework sequences. A further description of the Nanobody®, including humanization and/or camelization of Nanobody®, as well as other modifications, parts or fragments, derivatives or “Nanobody® fusions”, multivalent constructs (including some non-limiting examples of linker sequences) and different modifications to increase the half-life of the Nanobody® and their preparations can be found in e.g. WO 08/101985 and WO 08/142164.

“Domain antibodies”, also known as “Dabs” (the terms “Domain Antibodies” and “dAbs” being used as trademarks by the GlaxoSmithKline group of companies) have been described in e.g., EP 0368684, Ward et al. 1989 (Nature 341:544-546), Holt et al. 2003 (Trends in Biotechnology 21:484-490) and WO 03/002609, WO 04/068820, WO 06/030220, and WO 06/003388. Domain antibodies essentially correspond to the VH or VL domains of non-camelid mammalians, in particular human 4-chain antibodies. In order to bind an epitope as a single antigen binding domain, i.e., without being paired with a VL or VH domain, respectively, specific selection for such antigen binding properties is required, e.g. by using libraries of human single VH or VL domain sequences. Domain antibodies have, like VHHs, a molecular weight of approximately 13 to approximately 16 kDa and, if derived from fully human sequences, do not require humanization for e.g. therapeutic use in humans. It should also be noted that single variable domains can be derived from certain species of shark (for example, the so-called “IgNAR domains”, see e.g. WO 05/18629).

When an Fc-region is present in an antibody (any format; the Fc-region either naturally present or introduced by means of genetic engineering), antibody-dependent cellular cytotoxicity (ADCC) can be part of the antibody's action in that when the Fc-region is capable of binding to an Fcγ receptor (FcγR or FCGR) on the surface of an immune effector cell, the cell carrying the antibody's target can be killed or destroyed. When an antibody comprises a (naturally occurring or engineered) C1q binding site, complement-dependent cytotoxicity (CDC) can be part of the antibody's action. When an antibody comprises a (naturally occurring or engineered) Fc domain capable of binding to a specific receptor on phagocytic cells, antibody-dependent cellular phagocytosis (ADCP) can be part of the antibody's action. ADCC-, CDC-, and ADCP-inducing antibodies thus are included herein as means of effectuating pharmacological inhibition of a target of interest.

Alphabodies are also known as Cell-Penetrating Alphabodies and are small 10 kDa proteins engineered to bind to a variety of antigens.

Aptamers have been selected against small molecules, toxins, peptides, proteins, viruses, bacteria, and even against whole cells. DNA/RNA/XNA aptamers are single stranded oligonucleotides and are typically around 15-60 nucleotides in length, although longer sequences of 220 nt have been selected; they can contain non-natural nucleotides (XNA) as described for antisense RNA. A nucleotide aptamer binding to the vascular endothelial growth factor (VEGF) was approved by FDA for treatment of macular degeneration. Variants of RNA aptamers are spiegelmers are composed entirely of an unnatural L-ribonucleic acid backbone. A Spiegelmer of the same sequence has the same binding properties of the corresponding RNA aptamer, except it binds to the mirror image of its target molecule.

Peptide aptamers consist of one (or more) short variable peptide domains, attached at both ends to a protein scaffold, e.g. the Affimer scaffold based on the cystatin protein fold. Although not called aptamers, a type of further variation is described in e.g. WO 2004/077062 wherein e.g. 2 peptide loops are attached to an organic scaffold to arrive at a bicyclic peptide (which can be further multimerized). Phage-display screening of such bicyclic peptides to arrive at species binding with high-affinity to a target has proven to be possible in e.g. WO 2009/098450.

DARPins stands for designed ankyrin repeat proteins. DARPin libraries with randomized potential target interaction residues, with diversities of over 10∧12 variants, have been generated at the DNA level. From these, DARPins can be selected for binding to a target of choice with picomolar affinity and specificity or selectivity.

Affitins, or nanofitins, are artificial proteins structurally derived from the DNA binding protein Sac7d, found in Sulfolobus acidocaldarius. By randomizing the amino acids on the binding surface of Sac7d and subjecting the resulting protein library to rounds of ribosome display, the affinity can be directed towards various targets, such as peptides, proteins, viruses, and bacteria.

Anticalins are derived from human lipocalins which are a family of naturally binding proteins and mutation of amino acids at the binding site allows for changing the affinity and selectivity towards a target of interest. They have better tissue penetration than antibodies and are stable at temperatures up to 70° C.

Monobodies are synthetic binding proteins that are constructed starting from the fibronectin type III domain (FN3) as a molecular scaffold.

Affibodies are composed of alpha helices and lack disulfide bridges, and are based on the Z or IgG-binding domain scaffold of protein A wherein amino acids located in the parental binding domain are randomized. Screening for affibodies for specific or selective binding to a desired target typically is performed using phage display.

Intrabodies are antibodies binding and/or acting to intracellular target; this typically requires the expression of the antibody within the target cell, which can be accomplished by gene therapy/genetic modification involving introduction in a cell of a suitable genetic construct or vector comprising a suitable promoter (e.g. inducible, organ- or cell-specific, . . . ) operably linked to an intrabody coding sequence.

### Pharmacological Knock-Down of a Protein of Interest

Several technologies can be applied to cause pharmacological knock-down of a target protein or protein of interest. Outlined hereafter are the general principles of agents causing pharmacological knock-down of a target protein by means of inducing (proteolytic) degradation of that target protein.

A proteolysis targeting chimera, or PROTAC, is a chimeric polypeptidic molecule comprising a moiety recognized by an ubiquitin ligase and a moiety binding to a target protein. Interaction of the PROTAC with the target protein causes it to be poly-ubiquinated followed by proteolytic degradation by a cell's own proteasome. As such, a PROTAC provides the possibility of pharmacologically knocking down a target protein. The moiety binding to a target protein can be a peptide or a small molecule (reviewed in, e.g., Zou et al. 2019, Cell Biochem Funct 37:21-30). Other such target protein degradation inducing technologies include dTAG (degradation tag; see, e.g., Nabet et al. 2018, Nat Chem Biol 14:431), Trim-Away (Clift et al. 2017, Cell 171:1692-1706), chaperone-mediated autophagy targeting (Fan et al. 2014, Nat Neurosci 17:471-480) and SNIPER (specific and non-genetic inhibitor of apoptosis protein (IAP)-dependent protein erasers; Naito et al. 2019, Drug Discov Today Technol, doi:10.1016/j.ddtec.2018.12.002).

Lysosome targeting chimeras, or LYTACs, are chimeric molecules comprising a moiety binding to a lysosomal targeting receptor (LTR) and a moiety binding to a target protein (such as an antibody). Interaction of the LYTAC with the target protein causes it to be internalized followed by lysosomal degradation. A prototypic LTR is the cation-independent mannose-6-phosphate receptor (ciMPR) and an LTR binding moiety is e.g. an agonist glycopeptide ligand of ciMPR. The target protein can be a secreted protein or a membrane protein (see, e.g., Banik et al. 2019, doi.org/10.26434/chemrxiv.7927061.v1).

### Treatment/Therapeutically Effective Amount

The terms therapeutic modality, therapeutic agent, and agent are used interchangeably herein, and likewise relate to immunotherapeutic compounds or agents. All refer to a therapeutically active compound, to a combination of therapeutically active compounds, or to a therapeutically active composition (comprising one or more therapeutically active compounds).

“Treatment”/“treating” refers to any rate of reduction, delaying or retardation of the progress of the disease or disorder, or a single symptom thereof, compared to the progress or expected progress of the disease or disorder, or single symptom thereof, when left untreated. This implies that a therapeutic modality on its own may not result in a complete or partial response (or may even not result in any response), but may, in particular when combined with other therapeutic modalities (such as, but not limited thereto: surgery, radiation, etc.), contribute to a complete or partial response (e.g. by rendering the disease or disorder more sensitive to therapy). More desirable, the treatment results in no/zero progress of the disease or disorder, or single symptom thereof (i.e. “inhibition” or “inhibition of progression”), or even in any rate of regression of the already developed disease or disorder, or single symptom thereof. “Suppression/suppressing” can in this context be used as alternative for “treatment/treating”. Treatment/treating also refers to achieving a significant amelioration of one or more clinical symptoms associated with a disease or disorder, or of any single symptom thereof. Depending on the situation, the significant amelioration may be scored quantitatively or qualitatively. Qualitative criteria may e.g. by patient well-being. In the case of quantitative evaluation, the significant amelioration is typically a 10% or more, a 20% or more, a 25% or more, a 30% or more, a 40% or more, a 50% or more, a 60% or more, a 70% or more, a 75% or more, a 80% or more, a 95% or more, or a 100% improvement over the situation prior to treatment. The time-frame over which the improvement is evaluated will depend on the type of criteria/disease observed and can be determined by the person skilled in the art.

A “therapeutically effective amount” refers to an amount of a therapeutic agent to treat or prevent a disease or disorder in a subject (such as a mammal). In the case of cancers, the therapeutically effective amount of the therapeutic agent may reduce the number of cancer cells; reduce the primary tumor size; inhibit (i.e., slow down to some extent and preferably stop) cancer cell infiltration into peripheral organs; inhibit (i.e., slow down to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, e.g., be measured by assessing the duration of survival (e.g. overall survival), time to disease progression (TTP), response rates (e.g., complete response and partial response, stable disease), length of progression-free survival (PFS), duration of response, and/or quality of life.

The term “effective amount” or “therapeutically effective amount” may depend on the dosing regimen of the agent/therapeutic agent or composition comprising the agent/therapeutic agent (e.g. medicament or pharmaceutical composition). The effective amount will generally depend on and/or will need adjustment to the mode of contacting or administration. The effective amount of the agent or composition comprising the agent is the amount required to obtain the desired clinical outcome or therapeutic effect without causing significant or unnecessary toxic effects (often expressed as maximum tolerable dose, MTD). To obtain or maintain the effective amount, the agent or composition comprising the agent may be administered as a single dose or in multiple doses. The effective amount may further vary depending on the severity of the condition that needs to be treated; this may depend on the overall health and physical condition of the subject or patient and usually the treating doctor's or physician's assessment will be required to establish what is the effective amount. The effective amount may further be obtained by a combination of different types of contacting or administration.

The aspects and embodiments described above in general may comprise the administration of one or more therapeutic compounds to a subject (such as a mammal) in need thereof, i.e., harboring a tumor, cancer or neoplasm in need of treatment. In general a (therapeutically) effective amount of (a) therapeutic compound(s) is administered to the mammal in need thereof in order to obtain the described clinical response(s).

“Administering” means any mode of contacting that results in interaction between an agent (e.g. a therapeutic compound or immunotherapeutic compound or agent) or composition comprising the agent (such as a medicament or pharmaceutical composition) and an object (e.g. cell, tissue, organ, body lumen) with which said agent or composition is contacted. The interaction between the agent or composition and the object can occur starting immediately or nearly immediately with the administration of the agent or composition, can occur over an extended time period (starting immediately or nearly immediately with the administration of the agent or composition), or can be delayed relative to the time of administration of the agent or composition. More specifically the “contacting” results in delivering an effective amount of the agent or composition comprising the agent to the object.

### Combinations

The invention further relates to a combination of an inhibitor of SLC4A4 and an immunotherapeutic compound or agent. Alternatively, the invention relates to a combination of a composition, such as a pharmaceutically acceptable composition, comprising an inhibitor of SLC4A4; and of a composition, such as a pharmaceutically acceptable composition, comprising an immunotherapeutic compound or agent. In one embodiment thereto, the invention relates to a combination of an inhibitor of SLC4A4 and an immunotherapeutic compound or agent which is an immune checkpoint inhibitor.

In a further embodiment thereto, the invention relates to a combination of an inhibitor of SLC4A4 and an immunotherapeutic compound or agent which is a combination of two immune checkpoint inhibitors. In the latter case a further embodiment relates to a combination of a composition, such as a pharmaceutically acceptable composition, comprising an inhibitor of SLC4A4; of a composition, such as a pharmaceutically acceptable composition, comprising a first immune checkpoint inhibitor; and of a composition, such as a pharmaceutically acceptable composition, comprising a second immune checkpoint inhibitor.

The invention further relates to any composition comprising a combination of an inhibitor of SLC4A4 and an immunotherapeutic compound or agent as described hereinabove for use as a medicine or medicament. Alternatively, the invention relates to a medicine or medicament comprising a combination of an inhibitor of SLC4A4 and an immunotherapeutic compound or agent as described hereinabove. In one embodiment thereto, these combinations, compositions, medicines or medicaments are for use in treating or inhibiting cancer, or for use in inhibiting progression, relapse or metastasis of cancer. In one embodiment, the cancer is poorly responding to or resistant to immunotherapy or to therapy comprising an immunotherapeutic compound or agent.

The invention further relates to any composition comprising an inhibitor of Solute Carrier Family 4 member 4 (SLC4A4) for use in treating or inhibiting pancreatic cancer, or for use in inhibiting progression, relapse or metastasis of pancreatic cancer, of lung cancer, of glioblastoma, or of colorectal cancer. In any of the above aspects and embodiments, the tumor or cancer in particular is poorly responding to, resistant to, or refractory to immunotherapy or to therapy comprising an immunotherapeutic compound or agent.

Any of these combinations, compositions, medicines or medicaments may further be combined with another anti-cancer treatment or therapy such as surgery, radiation, chemotherapy etc.

“Combination”, “combination in anyway” or “combination in any appropriate way” as referred to herein is meant to refer to any sequence of administration of two (or more) therapeutic modalities, i.e. the administration of the two (or more) therapeutic modalities can occur concurrently in time or separated from each other by any amount of time; and/or “combination”, “combination in any way” or “combination in any appropriate way” as referred to herein can refer to the combined or separate formulation of the two (or more) therapeutic modalities, i.e. the two (or more) therapeutic modalities can be individually provided in separate vials or (other suitable) containers, or can be provided combined in the same vial or (other suitable) container. When combined in the same vial or (other suitable) container, the two (or more) therapeutic modalities can each be provided in the same vial/container chamber of a single-chamber vial/container or in the same vial/container chamber of a multi-chamber vial/container; or can each be provided in a separate vial/container chamber of a multi-chamber vial/container.

### Kits

The invention further relates to kits comprising a container or vial (any suitable container or vial, such as a pharmaceutically acceptable container or vial) comprising an inhibitor of SLC4A4 or comprising a composition comprising an inhibitor of SLC4A4; and optionally comprising a container or vial (any suitable container or vial, such as a pharmaceutically acceptable container or vial) comprising an immunotherapeutic compound or agent, such as an immune checkpoint inhibitor. One embodiment relates to kits comprising a container or vial (any suitable container or vial, such as a pharmaceutically acceptable container or vial) comprising an inhibitor of SLC4A4 or comprising a composition comprising an inhibitor of SLC4A4; and optionally: comprising a first immune checkpoint inhibitor or a composition comprising a first immune checkpoint inhibitor and comprising a container or vial (any suitable container or vial, such as a pharmaceutically acceptable container or vial) comprising a second immune checkpoint inhibitor or a composition comprising a second immune checkpoint inhibitor.

Alternatively, such kits are comprising a container or vial (any suitable container or vial, such as a pharmaceutically acceptable container or vial) comprising a combination of an inhibitor of SLC4A4 and an immunotherapeutic compound or agent (such as one or two immune checkpoint inhibitors) (see discussion on “combination in any way” on how such combination in a single container, e.g., vial can be defined). Other optional components of such kit include one or more diagnostic agents capable of predicting, prognosing, or determining the success of a therapy comprising one of the therapies according to the invention; use instructions; one or more containers with sterile pharmaceutically acceptable carriers, excipients or diluents [such as for producing or formulating a (pharmaceutically acceptable) composition of the invention]; one or more syringes; one or more needles; etc. In particular, such kits may be pharmaceutical kits.

Immune checkpoints antagonists or inhibitors as referred to herein include the cell surface protein cytotoxic T lymphocyte antigen-4 (CTLA-4), programmed cell death protein-1 (PD-1) and their respective ligands. CTLA-4 binds to its co-receptor B7-1 (CD80) or B7-2 (CD86); PD-1 binds to its ligands PD-L1 (B7-H10) and PD-L2 (B7-DC). Other immune checkpoint inhibitors include the adenosine A2A receptor (A2AR), B7-H3 (or CD276), B7-H4 (or VTCN1), BTLA (or CD272), IDO (indoleamine 2,3-10 dioxygenase), KIR (killer-cell immunoglobulin-like receptor), LAG3 (lymphocyte activation gene-3), NOX2 (nicotinamide adenine dinucleotide phosphate (NADPH) oxidase isoform 2), TIM3 (T-cell immunoglobulin domain and mucin domain 3), VISTA (V-domain Ig suppressor of T cell activation), SIGLEC7 (sialic acid-binding immunoglobulin-type lectin 7, or CD328) and SIGLEC9 (sialic acid-binding immunoglobulin-type lectin 9, or CD329).

In any of the above methods, embodiments, and kits, referring to two immune checkpoint inhibitors, in one embodiment these are each inhibiting a different immune checkpoint or a different immune checkpoint-ligand interaction. For instance, when an inhibitor of PD1 is selected as a first immune checkpoint inhibitor, the second immune checkpoint inhibitor could be an inhibitor of PDL1 or an inhibitor of PDL2. Such first and second immune checkpoint inhibitor are each inhibiting a different immune checkpoint protein. In a further non-limiting example, an inhibitor of PD1 is selected as a first immune checkpoint inhibitor, and as second immune checkpoint inhibitor an inhibitor different from an inhibitor of PDL1 and different from an inhibitor of PDL2 is selected, e.g. an inhibitor of CTLA-4 is selected. In this latter example, the first and second immune checkpoint inhibitor are not only each inhibiting a different immune checkpoint, but also each inhibiting a different immune checkpoint-ligand interaction.

In referring to genes or proteins herein, no distinction is made in the annotation. Thus, whereas for example the human Slc4A4 gene would be referred to as the SLC4A4 gene, the mRNA as SLC4A4 mRNA, and the protein as SLC4A4, such distinction is not, or not always, made hereinabove or hereinafter.

### Immunotherapy/Immunotherapeutic Compound or Agent

Immunotherapy in general is defined as a treatment comprising administration of an immunotherapeutic compound or agent that supports (including activation or reactivation) the body's own immune system to help fight a disease, more specifically cancer in the context of the current invention. Immunotherapeutic treatment as used herein refers to the reactivation and/or stimulation and/or reconstitution of the immune response of a mammal towards a condition such as a tumor, cancer or neoplasm evading and/or escaping and/or suppressing normal immune surveillance. The reactivation and/or stimulation and/or reconstitution of the immune response of a mammal in turn in part results in an increase in elimination of tumorous, cancerous or neoplastic cells by the mammal's immune system (anticancer, antitumor or anti-neoplasm immune response; adaptive immune response to the tumor, cancer or neoplasm).

Immunotherapeutic agents include antibodies, in particular monoclonal antibodies, employed as (targeted) anti-cancer agents include alemtuzumab (chronic lymphocytic leukemia), bevacizumab (colorectal cancer), cetuximab (colorectal cancer, head and neck cancer), denosumab (solid tumor's bony metastases), gemtuzumab (acute myelogenous leukemia), ipilumab (melanoma), ofatumumab (chronic lymphocytic leukemia), panitumumab (colorectal cancer), rituximab (Non-Hodgkin lymphoma), tositumomab (Non-Hodgkin lymphoma) and trastuzumab (breast cancer). Other antibodies include for instance abagovomab (ovarian cancer), adecatumumab (prostate and breast cancer), afutuzumab (lymphoma), amatuximab, apolizumab (hematological cancers), blinatumomab, cixutumumab (solid tumors), dacetuzumab (hematologic cancers), elotuzumab (multiple myeloma), farletuzumab (ovarian cancer), intetumumab (solid tumors), muatuzumab (colorectal, lung and stomach cancer), onartuzumab, parsatuzumab, pritumumab (brain cancer), tremelimumab, ublituximab, veltuzumab (non-Hodgkin's lymphoma), votumumab (colorectal tumors), zatuximab and anti-placental growth factor antibodies such as described in WO 2006/099698.

Immunotherapeutic agents of particular interest further include immune checkpoint inhibitors (such as anti-PD-1, anti-PD-L1 or anti-CTLA-4 antibodies; detailed hereinafter), bispecific antibodies bridging a cancer cell and an immune cell, dendritic cell vaccines, CAR-T cells, oncolytic viruses, RNA vaccines, and soon. Immunotherapy is a promising new area of cancer therapeutics and several immunotherapies are being evaluated preclinically as well as in clinical trials and have demonstrated promising activity (Callahan et al. 2013, J Leukoc Biol 94:41-53; Page et al. 2014, Annu Rev Med 65:185-202). However, not all the patients are sensitive to immune checkpoint blockade and sometimes PD-1 or PD-L1 blocking antibodies accelerate tumor progression. An overview of clinical developments in the field of immune checkpoint therapy is given by Fan et al. 2019 (Oncology Reports 41:3-14). Combinatorial cancer treatments that include chemotherapies can achieve higher rates of disease control by impinging on distinct elements of tumor biology to obtain synergistic antitumor effects. It is now accepted that certain chemotherapies can increase tumor immunity by inducing immunogenic cell death and by promoting escape in cancer immunoediting, such therapies are therefore called immunogenic therapies as they provoke an immunogenic response. Drug moieties known to induce immunogenic cell death include bleomycin, bortezomib, cyclophosphamide, doxorubicin, epirubicin, idarubicin, mafosfamide, mitoxantrone, oxaliplatin, and patupilone (Bezu et al. 2015, Front Immunol 6:187). Other forms of immunotherapy include chimeric antigen receptor (CAR) T-cell therapy in which allogenic T-cells are adapted to recognize a tumoral neo-antigen and oncolytic viruses preferentially infecting and killing cancer cells. Treatment with RNA, e.g. encoding MLKL, is a further means of provoking an immunogenic response (Van Hoecke et al. 2018, Nat Commun 9:3417), as well as vaccination with neo-epitopes (Brennick et al. 2017, Immunotherapy 9:361-371).

Further anti-tumor agents include those described in general terms in the sections “Inhibition of a target of interest”, and “Pharmacological knock-down of a protein of interest” included herein, and wherein the target or protein of interest can be any known anti-cancer target or protein.

### SLC4A4

Aliases of SLC4A4 provided in GeneCards® include Solute Carrier Family 4 Member 4; NBC1; Solute Carrier Family 4 (Sodium Bicarbonate Cotransporter), Member 4; Electrogenic Sodium Bicarbonate

Cotransporter 1; Na(+)/HCO3(−) Cotransporter; HNBC1; HhNMC; KNBC1; PNBC; NBCe1-A; NBCE1; KNBC; and NBC. The genomic locations for the SLC4A4 gene are chr4:71,062,646-71,572,087 (in GRCh38/hg38) and chr4:72,053,003-72,437,804 in GRCh37/hg19). The GenBank reference SLC4A4 mRNA sequences are known under accession nos. NM_001098484.3, NM_001134742.2, and NM_003759.4. SLC4A4 human shRNA lentiviral particles are offered for sale by e.g. Origene. Other SLC4A4 siRNA and shRNA products are available through e.g. Santa Cruz Biotechnology.

### PD1

Aliases of PD1 provided in GeneCards® include PDCD1; Programmed Cell Death 1; Systemic Lupus Erythematosus Susceptibility 2; PD-1; CD279; HPD-1; SLEB2; and HPD-L. The genomic locations for the PDCD1 gene are chr2:241,849,881-241,858,908 (in GRCh38/hg38) and chr2:242,792,033-242,801,060 (in GRCh37/hg19). The GenBank reference PD1 mRNA sequence is known under accession no. NM_005018.3. Approved PD1-inhibiting antibodies include nivolumab, pembrolizumab, and cemiplimab; PD1-inhibiting antibodies under development include CT-011 (pidilizumab) and therapy with PD1-inhibiting antibodies is referred to herein as α-PD-1 therapy or α-PD1 therapy. PD1 siRNA and shRNA products are available through e.g. Origene.

### PD-L1

Aliases of PD-L1 provided in GeneCards® include CD274, Programmed Cell Death 1 Ligand 1, B7 Homolog 1, B7H1, PDL1, PDCD1 Ligand 1, PDCD1LG1, PDCD1L1, HPD-L1, B7-H1, B7-H, and Programmed Death Ligand 1. The genomic locations for the PDCD1 gene are chr9:5,450,503-5,470,567 (in GRCh38/hg38) and chr9:5,450,503-5,470,567 (in GRCh37/hg19). The GenBank reference PD1 mRNA sequence is known under accession no. NM 001267706.1, NM 001314029.2 and NM 014143.4. Approved PD-L1-inhibiting antibodies include atezolizumab, avelumab, and durvalumab. PD-L1 siRNA and shRNA products are available through e.g. Origene.

### CTLA4

Aliases of CTLA4 provided in GeneCards® include Cytotoxic T-Lymphocyte Associated Protein 4; CTLA-4; CD152; Insulin-Dependent Diabetes Mellitus 12; Cytotoxic T-Lymphocyte Protein 4; Celiac Disease 3; GSE; Ligand And Transmembrane Spliced Cytotoxic T Lymphocyte Associated Antigen 4; Cytotoxic T Lymphocyte Associated Antigen 4 Short Spliced Form; Cytotoxic T-Lymphocyte-Associated Serine Esterase-4; Cytotoxic T-Lymphocyte-Associated Antigen 4; CELIAC3; IDDM12; ALPS5; and GRD4.

The genomic locations for the CTLA4 gene are chr2:203,867,771-203,873,965 (in GRCh38/hg38) and chr2:204,732,509-204,738,683 (in GRCh37/hg19). The GenBank reference CTLA4 mRNA sequences are known under accession nos. NM_001037631.3 and NM_005214.5. Approved CTLA4-inhibiting antibodies include ipilumab; CTLA4-inhibiting antibodies under development include tremelimumab; therapy with CTLA4-inhibiting antibodies is referred to herein as α-CTLA4 therapy. CTLA4 siRNA and shRNA products are available through e.g. Origene.

### EXAMPLES

### Example 1. Materials and Methods

### Animals

FVB, C57BL6/N and NMRI nu/nu athymic nude mice were purchased from Envigo. Rag2/OT-1 mice were purchased from Taconic. All mice used for tumor experiments were females between 8 and 12 weeks old. Housing and all experimental animal procedures were approved by the Institutional Animal Care and Research Advisory Committee of the KU Leuven.

### Cell Lines

The murine pancreatic ductal adenocarcinoma Panc02 cell line was kindly provided by Prof. B. Wiedenmann (Charité, Berlin). The cells were cultured in DMEM medium (Gibco) supplemented with 10% of bovine fetal serum (FBS) (Gibco) and 1% of penicillin/streptomycin (Pen/strep) antibiotic (Gibco). The murine pancreatic ductal adenocarcinoma KPC cell line was kindly provided by Hanahan's lab at the École Polytechnique Fédérale de Lausanne (EPFL) and it was generated from FVB mice carrying different genetic mutations P48Cre/KrasG12D/p53 LSL R172H. The cells were cultured in RPMI medium (Gibco) supplemented with 10% of bovine fetal serum (FBS) (Gibco) and 1% of penicillin/streptomycin (Pen/strep) antibiotic (Gibco). KP, KR158B and CMT-93 cells were cultured in DMEM medium (Gibco) supplemented with 10% of bovine fetal serum (FBS) (Gibco) and 1% of penicillin/streptomycin (Pen/strep) antibiotic (Gibco). All the cells were grown at 37° C. in a humidified 5% CO₂ incubator.

### Tumor Models

C57BL6/N were injected subcutaneously in the right flank with 1*10⁶ Panc02 cells (pancreatic cancer cell line), 2*10⁶ KP cells (lung cancer cell line), 0.8*10⁶ KR158B cells (glioblastoma cell line), or 5*106 CMT-93 cells (colorectal cancer cell line) in 200 μl. Tumor growth was monitored by measuring the perpendicular diameters of tumors every other day and mice were sacrificed at a humane endpoint. FVB mice were injected orthotopically in the head of the pancreas with 10′000 KPC cells in 20 μl. Body weight was monitored and mice were sacrificed at a humane endpoint. Alternatively, FVB mice were injected subcutaneously in the right flank with 0,5*10⁶ KPC cells in 200 μl. Tumor growth was monitored by measuring the perpendicular diameters of tumors every other day and mice were sacrificed at a humane endpoint. For immunotherapy treatment the mice were treated intraperitoneally (i.p.) with 10 mg/kg of either control IgG, anti-PD-1 (αPD-1), anti-PDL1 (αPDL1), or anti-CTLA-4 (αCTLA-4) antibodies (3×/week). For CD8 depletion mice were injected i.p. with an anti-CD8 antibody (10 mg/kg) 3 days before tumor inoculation and then 1 time per week. For in vivo slc4a4 inhibition, mice were treated with 15 mg/kg of 4,4′-Diisothiocyano-2,2′-stilbenedisulfonic acid (DIDS) i.p. bi-daily for a period of 10 days. Antibodies: Rat serum IgG (14131) (Sigma-Aldrich); Ultra-LEAF™ Purified PD-1anti-mouse (CD279) RMP1-14 (BioLegend); InVivoMAb anti-mouse CTLA-4 (CD152) (BioCell); InVivoMAb anti-mouse CD8α (BioCell); InVivoMab anti-mouse PD-L1 (B7-H1) (BioCell).

### Extracellular pH Measurements

Extracellular pH (pHₑₓₜ) measurements were performed directly in the medium of cultured cells using single-barreled H⁺-sensitive microelectrodes. pH microelectrodes were fabricated as described previously for double-barreled microelectrodes (Caroppo et al. 1997, J Physiol 499:763-771) but adopting the following modifications. Briefly, single-barreled microelectrodes were constructed from a piece of filament-containing aluminum silicate glass tubing of 1.5-mm outer diameter and 1.0-mm inner diameter (Hilgenberg, Malsfeld, Germany). Microelectrodes were pulled in a PE2 vertical puller (Narishige, Tokyo, Japan), silanized for 90 s in dimethyl-dichloro-silane vapor (Sigma, St. Louis, Missouri, U.S.A) and baked in the oven for 3 h at 140° C. Then the tip of the microelectrode was backfilled with a small amount of the proton ionophore cocktail (Hydrogen Ionophore II, Cocktail A; Sigma, St. Louis, Missouri, U.S.A) and its shaft was later filled with a buffer solution of pH 7.0. The reference electrode was an Ag/AgCl wire connected to ground. All microelectrodes were calibrated before and after the measurements with NaCl solutions containing a mixture of KH₂PO₄ and Na₂PO₄ to yield pH values between 6.8 and 7.8 and the measurement was rejected when the variation exceeded 10%. Average slope of the electrodes was 58.3±0.4 mV/pH unit (mean±SEM, n=14). To measure the extracellular pH in close proximity of cell membrane, the H⁺-sensitive microelectrode was mounted on a Leitz micromanipulator, connected to a dual-channel electrometer (WPI, CT) and to a strip-chart recorder (Kipp and Zonen, Holland).

### Spectrofluorimetric Measurements of pHi w and w/o HCO₃⁻ with Cary Eclipse

Cells, cultured on 12 mm coverslips as described above, were incubated with 4 μM 2′,7′-Bis-(2-Carboxyethyl)-5-(and-6)-Carboxyfluorescein, Acetoxymethyl Ester (BCECF-AM) in the dark for 1 hr at room temperature, in air. The coverslip is then placed inside a perfusion cuvette, where the cells are continually perfused. All the solutions are maintained at 37° and the cells are perfused always at the same rate. Using a Cary Eclipse spectrophotometer, cells were excited alternatively at 440 and 500 nm, while the BCECF fluorescence emission was collected at 535 nm. The resting pHi was measured with ringer w and w/o HCO₃⁻ at both pHe 6.7 and 7.4 (solutions composition reported in Table 1).

Intracellular pH was estimated from the ratio of BCECF fluorescence calibrated by using the K⁺ nigericin method. The cells were incubated with 4 μM BCECF-AM and 5 μM nigericin in a KCl rich medium for 1 h at RT. After the incubation, the cells were perfused with KCl medium at different pH values (6.7-7-7.4-8).

TABLE 1
Composition of Ringers used to measure pHi
The concentrations are expressed in mM. The pHs of solution
w/o bicarbonate is adjusted at 7.4 and 6.7 with NaOH., while
the different pHs of Rimger KCl with KOH.

| | Ringer w | Ringer w | | |
|---|---|---|---|---|
| | NaHCO₃ | NaHCO₃ | Ringer w/o | Ringer |
| | pHe 7.4 | pHe 6.7 | NaHCO₃ | KCl |
| NaCl | 120 | 138.5 | 135 | 20 |
| NaHCO₃ | 22 | 3.5 | / | / |
| KCl | 4.5 | 4.5 | 3 | 110 |
| CaCl₂ | 1 | 1 | 1.8 | 1 |
| MgCl₂ | 1 | 1 | / | / |
| MgSO₄ | / | / | 0.7 | 1 |
| KH₂PO₄ | / | / | 1 | / |
| Glucose | 11 | 12 | 11 | 18 |
| Hepes | / | / | 20 | 20 |

### Metabolite Analysis by LC-MS/MS

The cells were washed once with ice cold 0.9% NaCl solution. The metabolite extraction was performed using 80% methanol. After 5 minutes of incubation cells were scraped and collected in a new tube. Following a centrifugation at 20′000 g for 10 minutes at 4° C., the supernatant was transferred to a new vial for MS analysis. Pellet was used for protein quantification. 5 μl of each sample was loaded into a Dionex UltiMate 3000 LC System (Thermo Scientific Bremen, Germany) equipped with a C-18 column (Acquity UPLC-HSS T3 1. 8 μm; 2.1×150 mm, Waters) coupled to a Q Exactive Orbitrap mass spectrometer (Thermo Scientific) operating in negative ion mode. A step gradient was carried out using solvent A (10 mM TBA and 15 mM acetic acid) and solvent B (100% methanol). The gradient started with 0% of solvent B and 100% solvent A and remained at 0% B until 2 minutes post injection. A linear gradient to 37% B was carried out until 7 minutes and increased to 41% until 14 minutes. Between 14 and 26 minutes the gradient increased to 100% of B and remained at 100% B for 4 minutes. At 30 minutes the gradient returned to 0% B. The chromatography was stopped at 40 minutes. The flow was kept constant at 250 uL/min and the column was placed at 25° C. throughout the analysis. The MS operated in full scan −SIM (negative mode) using a spray voltage of 3.2 kV, capillary temperature of 320° C., sheath gas at 10.0, auxiliary gas at 5.0. For Full scan −SIM mode, AGC target was set at 1e6 using a resolution of 70.000, with a maximum IT of 256 ms. For the data analyses we integrated the peak areas using the Thermo XCalibur Quan Browser software (Thermo Scientific). Data were normalized on protein content or on the amount of interstitial fluid collected.

### Protein Extraction and Immunoblot

Whole cell protein extraction was performed using RIPA extraction Buffer (20 mM Tris HCl, 150 mM NaCl, 1% Triton X-100, 10% glycerol, 5 mM EDTA) supplemented with Complete Mini protease inhibitor (Roche) and PhosSTOP Phosphatase Inhibitor (Roche). Proteins (15-50 μg) were separated by Mini-PROTEAN. TGX Stain-Free™ Precast Gels (4568094, Bio-Rad) and transferred to nitrocellulose membrane rans-Blot Turbo Midi 0.2 μm Nitrocellulose (#1704159, Bio-Rad) using Trans-Blot. Turbo™ Transfer System (Bio-Rad). Nonspecific binding was blocked in PBS with 0.05% Tween-20 (TBST) containing 5% of bovine serum albumin. The following antibodies were used: Rabbit Anti-SLC4A4/NBC antibody (ab187511), anti-beta Tubulin antibody—Loading Control HRP (ab21058, Abcam), anti-Vinculin (V9131, Sigma-Aldritch) and appropriate HRP-conjugated secondary antibodies (Santa Cruz). Signal was visualized by Enhanced Chemiluminescent Reagents (ECL, Invitrogen) or West Femto by Thermo Scientific according to the manufacturer's instructions and acquired by a LAS 4000 CCD camera with ImageQuant software (GE Healthcare).

### In Vivo ³¹P-Magnetic Resonance Spectroscopy (MRS) and In Vivo Hyperpolarized 1-¹³C-Pyruvate MRS

MRS measurements were performed on Panc02 subcutaneous size-matched tumors on a dedicated 11.7T small animal MRI (BioSpec, Bruker BioSpin GmbH, Ettlingen, Germany). Animals were anesthetized by inhalation of isoflurane evaporated in air (2.5% in air for induction and 1%-2% in air for maintenance) and warmed using a circulating water system. Respiration rate was monitors using a pressure cushion (SA Instruments Inc., Stony Brook, NY, USA).

For in vivo pH measurements, 3-aminopropyl phosphonate (3-APP, Sigma-Aldrich) was administered intraperitonally (11 mmol/kg) 30 min before data acquisition. Experiments were carried out using a ¹H/³¹P-surface coil (2 cm in diameter, Bruker BioSpin GmbH, Ettlingen, Germany) positioned over the tumor mass.

For selection of the tumor region, the T2-weighted Rapid Acquisition with Relaxation Enhancement (RARE) sequences in two different slice orientations were performed. Localized ³¹P-NMR spectra were then acquired using a pulse sequence with tumor volume selection based on outer-volume suppression (Bandwith 10 kHz, α: 45°, Average: 4096, 2048 points, TR:500 ms, Acq Time : 34 min).

Using jMRUI v5, pHi and pHe measurements were calculated from the chemical shift between inorganic phosphate (Pᵢ) and α-ATP peaks and the 3-APP and α-ATP peaks, respectively, in the ³¹P spectra according to literature (Ojugo et al. 1999, NMR Biomed 12:498-504).

[1-¹³C] pyruvic acid (Cortecnet) solution (40 μl ) containing 15 mM of trityl radical OX63 (GE Healthcare) and 2 mM gadolinium was hyperpolarized at 1.4 K and 3.35 T using an HyperSense DNP polarizer (Oxford Instruments, Abingdon, UK). After 60 min, the polarized solution was rapidly dissolved in 3 mL of a heated buffer containing 100 mg/I EDTA, 40 mM HEPES, 30 mM NaCl, 80 mM NaOH, 30 mM of non-hyperpolarized unlabeled lactate. 250 μl of solution was quickly administered intravenously to the mice and ¹³C-spectra acquisition was started simultaneously.

Mice were scanned using a double tuned ¹H/¹³C-surface coil (RAPID Biomedical, Rimpar, Germany) designed with a tumor-shaped cavity of 12 mm in diameter. Anatomic T2-weighted images were used to assess tumor volume and to validate the position of the tumor in the coil. ¹³C-spectra were acquired every 3 seconds for 210 seconds using a single pulse sequence (Bandwidth: 50 kHz; α: 10°; 10000 points). Using homemade routines in MATLAB (Mathworks, Natick, MA, USA), Peak areas under the curve were measured for each repetition time and each time point. The integrated peak intensities of hyperpolarized ¹³C-pyruvate, ¹³C-lactate and total observed ¹³C-signal were then measured to calculate the lactate-to-pyruvate (Lactate/Pyruvate) and lactate-to-total carbon (Lactate/Total Carbon) ratios.

### Slc4a4 Silencing

Cancer cells were transduced with lentiviral vectors in a medium supplemented with 1 μg/ml of polybrene. Firstly, they were transduced with a vector containing Cas9 under the control of a doxycycline inducible promoter. Secondly, they were transduced with a vector containing a sgRNA targeting the Slc4a4 locus (GATGAATCGGATGCGTTCTG-1^{st} gRNA (SEQ ID NO:1) and GCCTCCAAAAGTGATGGCGT-2^{nd} gRNA (SEQ ID NO:4)) or a non-targeting control sgRNA (GAACAGTCGCGTTTGCGACT, SEQ ID NO:2). To guarantee that each cell is infected with a single Cas9 copy, we used a multiplicity of infection reaching approximately 30% of transduction. Transduced cells were selected with blasticidin (20 μg/ml) and puromycin (2 μg/ml), respectively. After selection, cells were treated for seven days with doxycycline (0.5 μg/mL) in order to induce Cas9 expression and following gene editing. Subsequently, cells were kept in doxycycline-free medium for at least seven days before performing any experiment. Gene silencing was confirmed by western blot analysis.

In order to target Slc4a4 in KR158B, KP and CMT-93 cells we used a nuclefection method. For this purpose, the Alt-R CRISPR-Cas9 cRNA (IDT) for Slc4a4 (GCGATGGAGCAAACCCCATG; SEQ ID NO:5) or a non targeting control and the Alt-R CRISPR-Cas9 tracRNA (IDT) were mixed in equimolar concentrations to have a final duplex concentration of 50 μM and the annealing was performed as follows: 95° C. 5 min; 90° C. 2 min; 85° C. 2 min; 80° C. 2 min; 75° C. 2 min; 70° C. 2 min; 65 ° C. 2 min; 60° C. 2 min; 55° C. 2 min; 50° C. 2 min; 45° C. 2 min; 40° C. 2 min; 35° C. 2 min; 30° C. 2 min; 25° C. inf. RNP complexes were then generated by incubating duplex RNA with Cas9 enzyme in a 3:1 ratio at RT for 20 minutes. Cancer cells were harvested, washed twice in PBS, and resuspended at a concentration of 50*10⁶/ml in P4 Nucleofector solution (P4 Primary Cell 4D-Nucleofector X kit L, Lonza). 5*10⁶ cancer cells were then incubated with the RNP complex RT for 2 minutes, transferred to the cuvette (P4 Primary Cell 4D-Nucleofector X kit L, Lonza), and electroporated with the program CM150 on a 4D-Nucleofector System (Lonza). The cells were then collected from the cuvette and dispensed into a 6-well plate containing pre-warmed cancer cell medium.

After 5 days, cells were sorted based on Slc4a4 expression. Briefly, cells were detached and washed with FACS buffer (PBS containing 2% FBS and 2 mM EDTA). Then, cells were incubated for 30 minutes at 4° C. with 100 nM binding nanobody for Slc4a4. After that, cells were washed with FACS buffer and stained for 30 minutes at 4° C. with viability dye (eFluor™ 450, 1:500) and anti-FLAG (L5 clone) (PE, 1:500). Cells were subsequently washed, resuspended in FACS buffer and sorted using BD FACSAria Fusion Cell Sorter. Data was analyzed by FlowJo (TreeStar).

### FACS Analysis

Mice were sacrifice by cervical dislocation and the tumor were harvested in cold PBS. Tumors were minced in alpha MEM (Lonza) containing 0,085 mg/ml Collagenase V (Sigma), 0,125 mg/ml Collagenase D (Roche) and 0,1 mg/ml Dispase (Gibco), and incubated in the same solution for 30 minutes at 37° C. The digested tissues were filtered using a 70-μm pore sized strainer and cells were centrifuged 5 minutes at 300 xg. Red blood cell lysis was performed by using a home-made red blood cell lysis buffer (150 mM NH4Cl, 0.1 mM EDTA, 10 mM KHCO3, pH 7.4. Single cells were resuspended in FACS buffer (PBS containing 2% FBS and 2 mM EDTA) and incubated for 15 minutes with Mouse BD Fc Block purified anti-mouse CD16/CD32 mAb (BD-Pharmingen) and stained for 30 minutes at 4° C. with the following antibodies: Fixable viability dye (eFluor™ 450 or eFluor™ 506, 1:500),CD45 (PE, 1:200), TCRβ (FITC, 1:300), CD4 (PercPcy5.5, 1:400), CD8 (APC-cy7, 1:300), IFNγ (PE-cy7, 1:100), Foxp3 (APC, 1:100), TBet1 (BV421, 1:50)-from BD Biosciences. Cells were subsequently washed and resuspended in FACS buffer before FACS analysis by a FACS Canto II (BD Biosciences). Data was analyzed by Flowio (TreeStar).

### T Cell Isolation and Activation

Naïve mouse T cells were isolated from spleen. Single cell suspension was generated by processing and filtering the cells through a 40-μm pore cell strainer in sterile PBS. Red blood cells were lysis was performed using Red Blood Cell (RBC) Lysis Buffer (Sigma-Aldrich). Total splenocytes were cultured in T cell medium—RPMI medium supplemented with 10% FBS, 1% Pen/Strep, 1% MEM Non-Essential Amino Acids (NEAA), 25 μm beta-mercaptoethanol and 1 mM Sodium Pyruvate (all Gibco) at 37° C. in a humidified 5% CO2 incubator. According to the experimental requirements, T cells were activated for 3 days by adding CD3/CD28 Dynabeads™ (Thermo Fisher Scientific) at a 1:1 bead-to-cell ratio and 30 U/mL rIL-2 (PeproTech).

OT-I T cells were isolated from OT-I mice. These mice have a monoclonal population of naïve T cell receptor (TCR) transgenic CD8+ T cells (OT-I T cells) that recognize the ovalbumin (OVA) “SIINFEKL” (SEQ ID NO:3) peptide. For activation of OT-I T cells, total splenocytes from OT-I mice were isolated and cultured for 3 days in T cell medium with 1 μg/ml SIINFEKL peptide (IBA—LifeSciences; SEQ ID NO:3) and 30 U/ml rIL-2 (PeproTech).

### T Cell Cytotoxicity Assay

10*10⁴ Panc02 OVA cancer cells labelled 1 μM carboxyfluorescein succinimidyl ester (CFSE; Thermo Fisher Scientific) were seeded in a 96 well round bottom plate. After the cells had attached, activated OT1 T cells were added to the plate at a 1:5 target:effector ratio. T cells and cancer cells were co-cultured in T cell medium alone or supplemented with 10 mM sodium lactate or 10 mM Lactic acid or with the needed amount of HCl to reach the same acidity induced by the lactic acid condition. After 24 hours cells were detached and stained with Fixable viability dye (eFluor™ 450, 1:500) and CD8 (APC-cy7, 1:300) antibody from BD Biosciences. Cells were subsequently washed and resuspended in FACS buffer before FACS analysis by a FACS Canto II (BD Biosciences). Data was analyzed by FlowJo (TreeStar).

The absolute number of cancer cells was obtained by adding to the samples precision counting beads (Biolegends) and then normalized for the cancer cells cultured alone.

### T Cells Proliferation Assay

After isolation (as described above) splenocytes were labelled 1 μM carboxyfluorescein succinimidyl ester (CFSE; Thermo Fisher Scientific) at RT for 10 minutes and then cultured for 3 days with CD3/CD28 Dynabeads™ (Thermo Fisher Scientific) in a medium composed by 1/3 of T cell medium and 2/3 of cancer cell conditioned medium. After 3 days cells were collected, washed and resuspended in FACS buffer before FACS analysis by a FACS Canto II (BD Biosciences). Data was analyzed by FlowJo (TreeStar).

### Statistical Analysis

All statistical analyses were performed using GraphPad Prism software. Statistical significance was calculated by two-tailed paired or unpaired t test on two experimental conditions and one/two-way ANOVA test when more than two experimental groups were compared. Additionally, multiple comparisons were performed and, in all cases, an adjusted p-value of <0.05 was considered statistically significant (*<0,05; **<0,01; ***<0,001; ****<0,0001). All the results are shown as mean±SEM.

### Example 2. Deletion of slc4a4 in Pancreatic Cancer Cells Affects pH and Lactate Levels

The impact of inhibition of slc4a4 on the acidification and immune modulation of the tumor micro-environment (TME) was tested. Murine pancreatic cancer cells (Panc02) were generated that are deficient for Slc4a4 via the inducible CRISPR/Cas9 system (Slc4a4-knock-down or Slc4a4-KD Panc02 cells). As control, a non-targeting gRNA was used and these cells are denoted herein as NT Panc02 cells. A more than 80% reduction of slc4a4 protein was observed when comparing NT- vs. Slc4a4-KD-Panc02 cells (FIG. 1A). We opted for a doxycycline-inducible system for the expression of Cas9 in order to avoid potential immunogenic reactions against Cas9. Moreover, in order to confirm the knock-down (KD) from a functional point of view we measured the uptake of bicarbonate and we could observe a significant reduction in the Slc4a4-KD cells (FIG. 1B). Firstly, we performed an extensive analysis of the pH dynamics in vitro. We found that the deletion of Slc4a4 in Panc02 cells led to a slight decrease in the intracellular pH (pHᵢ) and to a reduction of the extracellular acidic levels (FIGS. 1C and 1D). These results were further confirmed in a second murine PDAC cell line, the KPC (P48:Cre; Kras^{G12D}; p53^{LSL.R172H}) model, where the KD of the transporter caused a reduction in the pHᵢ and an increase in the extracellular pH(pHₑ) (FIGS. 1E and 1F). Moreover, the deletion of Slc4a4 did not cause any change in cell proliferation, cell cycle distribution and apoptosis. To assess the effect of slc4a4 knock-down on the metabolism of the cell, we performed an in vitro metabolic characterization of Slc4a4-KD cancer cells by Liquid chromatography-Mass spectrometry (LC-MS). Our analysis revealed that Slc4a4 deletion reduces glycolysis in cancer cells as indicated by a reduction of both intracellular (54071±6852 A.U./μg in Slc4a4-KD vs. 111098±21761 A.U./μg in NT, p<0.05) and extracellular (495751±38845 in A.U./μg Slc4a4-KD vs. 798922±146610 A.U./μg in NT, p<0.05) levels of lactate, suggesting a diminished activity of lactate dehydrogenase A (LDHA), the enzyme involved in the conversion of pyruvate into lactate (FIGS. 2G and 2H). Altogether, these data indicate that beside the direct effect on limiting bicarbonate absorption, the metabolic rewiring upon Slc4a4 inhibition can also reduce the extracellular acidity through the reduction of lactate levels.

### Example 3. Slc4a4-KD Reduces Tumor Burden in Different in Vivo Pancreatic Cancer Models

The impact of Slc4a4 inhibition on tumor progression was next tested. To this aim, we subcutaneously implanted Panc02 tumors in immunocompetent mice observing that the deletion of the transporter in the cancer cell compartment reduced tumor growth (45% reduction compared to NT cells) (FIGS. 2A-C). The same decrease was also observed when the cells were injected orthotopically in the head of the pancreas instead that subcutaneously (FIG. 2D). Moreover, we could further confirm the same phenotype with a second gRNA directed against Slc4a4 (FIGS. 2 E-F).

Our in vivo data were further validated using the clinically relevant KPC model, which fully recapitulates the metabolic and histopathological features of human PDAC (Lee et al. 2016, Curr Protoc Pharmacol 73:14.39.1-14.39.20). In this setting, the effects of Slc4a4-KD were more pronounced, with a reduction in tumor growth of almost 90% (0,06±0,02 gr in Slc4a4-KD vs. 0,76±0,33 gr in NT, p<0.05), and a decrease in the number of mesenteric metastasis (0,9±0,7 in Slc4a4-KD vs. 4,5±2 in NT, p<0.05) (FIGS. 2G-J). Moreover, also in this case we could confirmed our results with a 2^{nd} gRNA directed against Slc4a4. Interestingly, Slc4a4-KD and NT cells did not show any difference in their proliferation index in vitro, suggesting that the reported reduction of tumor growth is due to a non-cell autonomous effect.

To confirm the observed metabolic alterations also in vivo, we measured both intracellular and extracellular pH by the aid of the ³¹P magnetic resonance spectroscopy (MRS). In size-matched tumors (FIG. 3A) we could observe a trend towards a reduced pHᵢ and an increase in the pHₑ towards a more alkaline extracellular space in the Slc4a4-KD tumors compared to their NT controls (6,6±0,3 in Slc4a4-KD vs. 5,9±0,4 in NT, p<0.05) (FIGS. 3B-D). Of note no difference in perfusion were observed. Moreover, based on differences in lactate levels observed in vitro, we measured via LC/MS the concentration of lactate in the extracellular fluid of the tumors and we could prove that also in vivo slc4a4 deletion lead to a reduction of the extracellular lactate both in the Panc02 subcutaneous model and in the KPC orthotopic model (FIGS. 3E-F). To further understand the origin of this difference we performed an in vivo experiment in which mice were treated with hyperpolarized ¹³C-pyruvate. Taking advantage of measurements done by nuclear magnetic resonance, we could assess, in real time, the transformation of polarized pyruvate into lactate which could be considered a readout of lactate dehydrogenase A (LDHA) activity. In this setting, we observed in the Panc02 model a decrease in the intratumoral lactate levels expressed as lactate to pyruvate ratio (1,7±0,3 in Slc4a4-KD vs. 1,6±0,5 in NT, p<0.05) and we confirmed that the reduction of lactate in the extracellular space is a direct consequence of a decreased LDHA activity (FIGS. 3G-H).

### Example 4. Reduced Tumor Growth of Slc4a4-KD Cancer Cells is Mediated by CD8 T Cells

The analysis of the immune infiltrate of Slc4a4-KD Panc02 tumors by flow cytometry revealed an increase in CD8⁺ T cell infiltration, (5,9±0,7 in Slc4a4-KD vs. 3,4±1,2 in NT, p<0.05), with a higher CD8⁺/CD4⁺ T cells ratio (1,2±0,17 in Slc4a4-KD vs. 0,7±0,21 in NT, p<0.05) (FIGS. 4A,C). A deeper analysis of the CD8⁺ T cell subset showed an augmented expression of the activation marker CD69 (1023±50 in Slc4a4-KD vs. 836±73 in NT MFI, p<0.05) and an increased secretion of the effector cytokine IFNγ (1039±398 in Slc4a4-KD vs. 567±188 in NT MFl, p<0.05) (FIG. 4B) indicating that CD8⁺ T cells were not only quantitatively more but also more active in the Slc4a4-KD tumors. Moreover, the examination of the CD4⁺ T cells in the Slc4a4-KD tumors showed no differences in their infiltration number and no differences in regulatory T cells, as indicated by the expression of Foxp3 (FIG. 4A). Furthermore, the same immune-phenotype was also confirmed in the KPC orthotopic model, were we also observed a strong increase in the CD8⁺ T cell infiltration reaching almost 10% of CD8⁺ cells out of alive cells in tumors that normally displays approximately 1-2% infiltration (9,6±3,6 in Slc4a4-KD vs. 1,7±1,9 in NT, p<0.05), in the IFNγ production (7470±3171 in Slc4a4-KD vs. 2401±1090 in NT MFI, p<0.05) and in the CD8⁺/CD4⁺ T cells ratio (1,5±0,5 in Slc4a4-KD vs. 0,5±0,3 in NT, p<0.05) (FIGS. 4D-F).

The increased activation of CD8⁺ T cells in Slc4a4-KD tumors was further corroborated by in vitro cytotoxic assays that were performed taking advantage of the OT-I T cell system (CD8⁺ T cells that recognize the OVA₂₅₇₋₂₆₄ immunogenic “SIINFEKL” peptide (SEQ ID NO:3) associated with MHC class I molecule H-2Kb). When ovalbumin-expressing cancer cells, presenting the immunogenic ovalbumin peptide SIINFEKL (SEQ ID NO:3) in MHC I, were co-cultured with OT-I T cells, we observed that OT-I T cells were able to kill more Slc4a4-KD cancer cells as compared to NT cells (FIG. 4G). Interestingly when the same assay was performed using a medium acidified with lactic acid or HCl we could not observe the difference in cancer cell killing. While the supplementation with sodium lactate did not affect the results (FIG. 4G) indicating a role of pH in the different killing ability demonstrated by T cells co-cultured with Slc4a4-KD cells. Additionally, CD8⁺ T cells grown in conditioned medium derived from Slc4a4-KD cancer cells displayed a more robust proliferation in vitro (1,74±0,04 in Slc4a4-KD vs. 1,68±0,02 in NT, p<0.05) (FIG. 4H). These results, in line with our metabolic data on pH and lactate metabolism, argue that Slc4a4-KD cancer cells alter the extracellular medium composition in a way to favor T cell proliferation and activation.

To further confirm that the observed tumor growth reduction in Slc4a4-KD tumors is due to the enhanced immune response (and, more specifically, by CD8⁺ T cell cytotoxicity), we injected Slc4a4-KD Panc02 cancer cells in immunodeficient mice or in WT animals depleted of CD8⁺ T cells by means of a CD8-specific depleting antibody. In both cases the difference in the tumor growth in Slc4a4-KD and NT was abolished (FIGS. 4I-J). Furthermore, the same phenotype was observed also in KPC tumor depleted of CD8⁺ T cells (FIG. 4K). These data underline the involvement of the adaptive immune response, and not a proliferative defect of cancer cells, in the observed tumor reduction of Slc4a4-KD tumors.

### Example 5. Slc4a4 Deletion Improves the Efficacy of the Immunotherapy Treatment

We have shown that the deletion of Slc4a4 in pancreatic cancer cells reinvigorates the anti-tumor immune response by the recruitment and activation of CD8+ T cells, ultimately leading to the inhibition of tumor growth. Based on these results, we investigated whether combination of Slc4a4 targeting and immunotherapy could achieve a synergistic effect. For this purpose, Slc4a4-KD and NT control tumors (KPC or Panc02) were treated with anti-PD-1 and anti-CTLA-4 antibodies. Treatment was given in 6 injections spread in two weeks, from the moment the tumor was established. In the subcutaneous setting with Panc02 tumors, this combinational treatment resulted in a synergistic effect with the Slc4a4 deletion leading to a static disease (FIGS. 5A-C). The same treatment in the orthotopic KPC model had an even more impressive result increasing dramatically mice survival when Slc4a4 was deleted in cancer cells. In particular, the control group that received two weeks of immunotherapy treatment had an average survival of 32 days dropping all dead by day 44, therefore with a mild effect of the treatment with immune checkpoint blockers (median survival of 26 days in NT IgG vs 32 days in NT αPD-1+αCTLA-4) (FIG. 5D). On the contrary, Slc4a4-KD tumors bearing mice treated with immune checkpoint blockers were all alive and perfectly active at day 80 when we decided to stop the experiment for further analysis. The necropsy did not reveal any sign of tumor or presence of metastasis indicating a complete tumor regression. Deletion of Slc4a4 alone increased the survival as well (median survival of 26 days in NT IgG vs 44,5 days in Slc4a4-KD IgG, FIG. 5D).

The synergy between Slc4a4-knock down in tumors and immune checkpoint inhibitors extends to other conditions and cancers. Indeed, in the orthotopic KPC pancreatic cancer model, it is sufficient to combine Slc4a4-knock down in tumor cells with a single immune checkpoint inhibitor, as is depicted in FIG. 7A for the immune checkpoint inhibitor anti-PD-1. Moreover, in this model, mice are protected against re-challenge of subcutaneously (i.e. remotely) injected KPC tumor cells, as is evidenced in FIG. 7B.

In addition, using a model of glioblastoma cancer, FIG. 8 shows that this cancer is refractive to immune checkpoint inhibitor therapy (anti-PDL1), and that Slc4a4-knock down in tumor cells sensitized the tumor to anti-PDL1 treatment.

Furthermore in addition, using a lung cancer model, FIG. 9A shows that that this cancer is poorly responding to immune checkpoint inhibitor therapy (anti-PDL1), and that Slc4a4-knock down in tumor cells sensitized the tumor to anti-PDL1 treatment. Using the same model, FIG. 9B shows that that this cancer is poorly responding to another immune checkpoint inhibitor therapy (anti-CTLA-4), and that Slc4a4-knock down in tumor cells sensitized the tumor to anti-CTLA-4 treatment.

Other cancer models that are being investigated are the KPC pancreatic cancer model in which Slc4a4-knock down in tumor cells is combined with anti-CTLA-4 immune checkpoint inhibitor therapy, and a colorectal cancer model in which Slc4a4-knock down in tumor cells is combined with anti-CTLA-4 immune checkpoint inhibitor therapy.

### Example 6. Systemic Administration of a slc4a4 Inhibitor Reduces Pancreatic Tumor Growth

We next explored the therapeutic potential of a commercially available pan-inhibitor for the bicarbonate transporters, 4,4′-diisothiocyano-2,2′-stilbenedisulfonic acid (DIDS). Although, this molecule is not a specific inhibitor of Slc4a4, our analysis identified Slc4a4 as the most expressed bicarbonate importer and exclusively expressed in PDAC epithelial cells, which renders it plausible that this treatment should effectively target Slc4a4 in cancer cells in a selective manner, thus mimicking our genetic approach.

Mice bearing an orthotopic KPC tumor were treated bi-daily with DIDS for 10 days from the moment the tumors were established. Consistent with the data obtained with the genetic deletion of Slc4a4, this treatment led to a reduction in tumor growth in wild-type (control, NT) tumors. On the other hand, no differences in tumor reduction was found on Slc4a4-KD tumors upon treatment suggesting that the effect of DIDS is, at least mainly, due to the inhibition of Slc4a4 rather than the general inhibition of bicarbonate transporters or other unrelated targets (FIGS. 6A,B).

Moreover, a further analysis of the immune infiltrate via flow cytometry of the WT tumors showed that the inhibitor treatment recapitulates also the immune phenotype induced by the deletion of Slc4a4. In fact, we could observe an increase of the CD8⁺ T cells infiltration and in particular of their IFNγ expression, an increase of the CD8⁺/CD4⁺ T cell ratio, and again no differences in the number of CD4⁺ T cells and Treg (FIGS. 6C-E).

### Example 7. Slc4A4 Inhibiting Antibodies.

Immunoglobulin single variable domain (ISVD) antibodies were raised against slc4a4 protein and preliminary results indicated some of the ISVDs to be capable of inhibiting slc4a4 activity.

ISVDs are molecules wherein the antigen binding site is present on, and formed by, a single immunoglobulin domain. This sets immunoglobulin single variable domains apart from “conventional” immunoglobulins (or conventional antibodies) or their fragments, wherein two immunoglobulin domains, in particular two variable domains, interact to form an antigen binding site.

1. A method of treating or inhibiting cancer, or inhibiting the progression, relapse or metastasis of cancer, the method comprising:
administering to the cancer an inhibitor of Solute Carrier Family 4 member 4 (SLC4A4),
wherein the cancer poorly responds to or is resistant to immunotherapy or to therapy comprising an immunotherapeutic compound or agent.

2. The method according to claim 1, wherein the cancer is pancreatic cancer.

3. The method according to claim 1, the method further comprising immunotherapy.

4. The method according to claim 1, wherein the inhibitor of SLC4A4 is a specific inhibitor of SLC4A4.

5. The method according to claim 4, wherein the specific inhibitor of SLC4A4 is a DNA nuclease specifically knocking out or disrupting SLC4A4, an RNase specifically targeting SLC4A4, or an inhibitory oligonucleotide specifically targeting SLC4A4.

6. The method according to claim 4, wherein the specific inhibitor of SLC4A4 is a pharmacological inhibitor specifically inhibiting SLC4A4 and is selected from the group consisting of a polypeptide comprising an immunoglobulin variable domain, a monoclonal antibody or a fragment thereof, an alpha-body, a nanobody, an intrabody, an aptamer, a DARPin, an affibody, an affitin, an anticalin, a monobody, a bicyclic peptide, a PROTAC, or a LYTAC.

7. The method according to claim 3, wherein the immunotherapy comprises therapy with one or two immune checkpoint inhibitors.

8. The method according to claim 7, wherein the two immune checkpoint inhibitors are each inhibiting a different immune checkpoint or a different immune checkpoint-ligand interaction.

9. (canceled)

10. (cancelled)

11. A combination of an inhibitor of Solute Carrier Family 4 member 4 (SLC4A4) and an immunotherapeutic compound or agent.

12. A composition comprising the combination of claim 11.

13. The combination of claim 11, wherein the immunotherapeutic compound or agent is at least one immune checkpoint inhibitor.

14. The combination of claim 11, wherein the combination is a medicine.

15. The combination of claim 11, wherein the combination inhibits the progression, relapse or metastasis of a cancer.

16. The combination of claim 15, wherein the cancer is poorly responding to or resistant to immunotherapy or to therapy comprising an immunotherapeutic compound or agent.


SEQUENCE LISTING (5 sequences)

SEQ ID NO: 1 - DNA, 20 bp, Artificial Sequence
  Slc4a4 locus targeted by sgRNA1
gatgaatcggatgcgttctg

SEQ ID NO: 2 - DNA, 20 bp, Artificial Sequence
  control locus targeted by sgRNA
gaacagtcgcgtttgcgact

SEQ ID NO: 3 - protein, 8 aa, Artificial Sequence
  ovalbumin peptide
SIINFEKL

SEQ ID NO: 4 - DNA, 20 bp, Artificial Sequence
  Slc4a4 locus targeted by sgRNA2
gcctccaaaagtgatggcgt

SEQ ID NO: 5 - DNA, 20 bp, Artificial Sequence
  Alt-R CRISPR-Cas9 cRNA
gcgatggagcaaaccccatg